(19) United States
(12) Patent Application Publication
(10) Pub. No.: US 2022/0026429 A1
(43) Pub. Date: Jan. 27, 2022

(54) COMPOSITIONS AND METHODS OF ALTERING A NUCLEIC ACID WITH RIBONUCLEASE

(71) Applicant: The Research Foundation for the State University of New York, Albany, NY (US)

(72) Inventors: Kenneth Anders Halvorsen, Glenmont, NY (US); Arun Richard Chandrasekaran, Albany, NY (US)

(21) Appl. No.: 17/381,836

(22) Filed: Jul. 21, 2021

Related U.S. Application Data
(60) Provisional application No. 63/054,761, filed on Jul. 21, 2020.

(51) Int. Cl. G01N 33/573; G01N 27/447

(52) U.S. Cl. CPC G01N 33/573; G01N 2333/922; G01N 27/447

(57) ABSTRACT

The present disclosure is directed to a polynucleotide capable of signaling under preselected conditions. For example, the present disclosure relates to a method of reconfiguring a nucleic acid or polynucleotide, including: contacting a deoxyribonucleic acid (DNA) nanoswitch and nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the nucleic acid is ribonucleic acid (RNA) and the biological specimen includes one or more ribonucleases, the first conformation changes to a second conformation characterized as open; processing the mixture under conditions sufficient to separate the first conformation, and when present, the second conformation; and reacting the first conformation, and when present, the second conformation with an indicator under conditions sufficient to form a signal.

### CROSS-REFERENCE TO RELATED APPLICATIONS

The present disclosure claims priority or the benefit under 35 U.S.C. § 119 of U.S. provisional application No. 63/054,761 filed Jul. 21, 2020, entirely incorporated by reference.

### STATEMENT OF GOVERNMENTAL SUPPORT

This invention was made with governmental support under grant No. GM124720 awarded by the National Institutes of Health. The government has certain rights in this invention.

### FIELD OF THE INVENTION

The present disclosure relates to the area of molecular biology and to nucleic acid compositions, polynucleotides, nucleic-acid complexes, and methods of use thereof. More specifically, the present disclosure relates to methods of altering a nucleic acid, polynucleotide, or nucleic-acid complex conformation with one or more ribonucleases. In embodiments, the present disclosure relates to methods for nanoswitch conformational change by contact with one or more ribonucleases.

### BACKGROUND

Ribonucleases or RNases are a type of enzyme or nuclease that catalyzes the degradation of ribonucleic acid (RNA) into smaller components. Two types of ribonucleases include endoribonucleases and exoribonucleases characterized by EC (Enzyme Commission) numbers: EC 2.7 and EC 3.1. RNases are important in many biological processes including neurotoxicity, genome replication and maintenance, angiogenic activity, immune-suppressivity and antitumor activity. For examples, in retroviruses such as HIV1, an RNase H activity associated with the viral reverse transcriptase is required for replication, making RNase H inhibitors potential drugs for acquired immune deficiency syndrome (HIV/AIDS). RNases are also potential biomarkers for neoplastic diseases such as pancreatic cancer and in cystic fibrosis. In a laboratory setting, RNases are important for some molecular biology protocols, but can also be the source of frustrating contaminations that degrade biological RNA samples. Detection of RNases and their inhibition have therefore become increasingly important, and various RNase detection kits are commercially available. Early methods developed to determine RNase activity include renaturation gel assays, high-performance liquid chromatography (HPLC), colorimetry, and fluorometry. The inventors have found that these methods are deficient and suffer from limitations such as complexity, high-cost, and low sensitivity, that spurred more recent detection approaches using catalytic hairpin assembly, gold nanoparticle conjugates, magnetic nanoparticles, DNA walkers and the like. The inventors have found these subsequent assays have higher sensitivity but problematically include multiple wash steps, additional amplification, indirect quantitation, and specific equipment for readout.

DNA nanoswitches are versatile nucleic acid complexes typically including a nucleic acid molecule, either single- or double-stranded, which is modified to contain segments of nucleic acids in a manner that would not otherwise exist in nature, which assume a linear (or open) conformation or a looped (or closed) formation depending upon one or more predetermined conditions such as the presence of an oligonucleotide such as a DNA or RNA oligonucleotide. DNA nanoswitches have been described in U.S. Patent Publication No. 2018/0223344 entitled Compositions and Methods for Analyte Detection Using Nanoswitches to Chandrasekaran et al., (herein entirely incorporated by reference) however it has not been heretofore contemplated to alter the conformation of a DNA nanoswitch as described herein such that the DNA nanoswitch is combined with a nucleic acid-of-interest to form a DNA nanoswitch-nucleic acid complex suitable for providing a signal or express a code as described herein, such as when contacted with a ribonuclease.

There is a continuing need for methods of detecting RNase, RNase activity, molecules having RNase activity, and molecules that inhibit RNase activity.

### SUMMARY

The present disclosure relates to a method of reconfiguring a nucleic acid complex in accordance with the present disclosure, which is useful for, inter alia, detecting RNase, RNase activity, molecules having RNase activity, and molecules that inhibit RNase activity. In some embodiments, the present disclosure relates to a method of reconfiguring a nucleic acid complex including: contacting a deoxyribonucleic acid (DNA) nanoswitch and a first nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the first nucleic acid is ribonucleic acid (RNA) and the biological specimen comprises one or more ribonucleases, the first conformation changes to a second conformation characterized as open; processing the mixture under conditions sufficient to separate the first conformation, and when present, the second conformation; and reacting the first conformation, and when present, the second conformation with an indicator under conditions sufficient to form a signal. In embodiments, the first nucleic acid is deoxyribonucleic acid or ribonucleic acid. In embodiments, the first nucleic acid is characterized as an oligonucleotide or polynucleotide having a preselected length. In embodiments, the first nucleic acid binds to the deoxyribonucleic acid (DNA) nanoswitch to form a first conformation including a loop, such as a loop having a preselected size.

In embodiments, the present disclosure includes a method of reconfiguring a polynucleotide including: contacting a deoxyribonucleic acid (DNA) nanoswitch and a first ribonucleic acid to form a DNA nanoswitch-nucleic acid complex having a first conformation, wherein the first conformation is characterized as locked; contacting the DNA nanoswitch-nucleic acid complex with a biological specimen comprising one or more ribonucleases to change the first conformation to a second conformation within a mixture, processing a mixture under conditions sufficient to separate the first conformation and the second conformation; and contacting the first conformation and second conformation with an indicator under conditions sufficient to form a signal. In embodiments, the signal is predetermined to show a presence or absence of ribonuclease. In embodiments, the signal is predetermined to indicate a code.

In some embodiments, the present disclosure includes a polynucleotide or polynucleotide complex, including: a DNA nanoswitch-nucleic acid complex including a deoxyribonucleic acid (DNA) nanoswitch and a first oligonucleotide, wherein the DNA nanoswitch-nucleic acid complex has a first conformation characterized as locked, and a second conformation characterized as open when in a presence of ribonuclease. In embodiments, the first oligonucleotide is a first nucleic acid of the present disclosure such as an RNA.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flowchart of a method of reconfiguring a nucleic acid in accordance with the present disclosure.

FIG. 2A shows the design and operation of a two-state DNA nanoswitch. A double stranded DNA is made with a single-stranded scaffold, complementary backbone oligos, and detector strands that can be addressably inserted at different locations. Addition of the key oligonucleotide binds the overhangs of the two detector regions ‘a’ and ‘b’ thereby forming a loop. This conformational change can be read out using gel electrophoresis.

FIG. 2B shows the sequence specificity of DNA nanoswitches. An agarose gel showing the sequence specificity of the nanoswitch. Switch A turns on only in the presence of key oligonucleotide A and switch B turns on only in the presence of key oligonucleotide B with no background detection of the incorrect strand.

FIG. 2C shows the loop size conformation of the on state. The left panel shows detector positions on the nanoswitch. The middle panel shows the combination of two positions, which gives different loop sizes on recognition of the key oligonucleotide. The right panel shows that different loop sizes can be identified using a gel read out. Larger loop sizes provide a shorter read-out time.

FIG. 2D shows data relating to the limit of detection using nanoswitches.

FIGS. 3A-3C are schematic illustrations of a DNA nanoswitch and DNA nanoswitch nucleic acid complex design and operation in accordance with embodiments of the present disclosure. FIG. 3A is a schematic illustration of a DNA nanoswitch-nucleic acid complex locked in a looped conformation with a pre-hybridized RNA lock strand or first nucleic acid. On the addition of RNase H, the lock strand is digested, resulting in unlooping of the nanoswitch to the open state. FIG. 3B is a schematic illustration of a mechanism of cleavage of the RNA lock strand by RNase H and release of the DNA latches or detector strands in accordance with the present disclosure. FIG. 3C is a schematic illustration depicting a DNA nanoswitch (1) locked by an RNA lock strand into a looped conformation (2). RNase H causes cleavage of the RNA lock, causing the nanoswitch to unlock and reconfigure into the linear state (3). This conformational change can be readout on an agarose gel to detect the presence of RNase H (inset).

FIGS. 4A-4G include graphs, gel readouts, and schematic illustrations of an RNase H assay of the present disclosure. FIG. 4A is a sensitivity plot showing nanoswitch unlocking with different enzyme concentrations (gel image shown as inset). FIG. 4B is a schematic illustration of detection of RNase H in 10% FBS. FIG. 4C is a schematic illustration of detection of RNase H in human (HeLa) and murine (C2C12) cell lysates.

FIG. 4D includes a series of gel readouts indicting the nanoswitch-based RNase assay works at a range of temperatures. FIG. 4E shows activity of different RNases (H, T, I_{f} and A) in the nanoswitch assay. FIG. 4F depicts inhibition efficiency of kanamycin on RNase H activity. FIG. 4G depicts a rapid readout that can be obtained by mixing nanoswitches with the sample containing RNase H and a quick gel run.

FIGS. 5A-5E depict schematic illustrations, and gel readouts relating to multi-input DNA nanoswitch of the present disclosure and information processing in accordance with embodiments, of the present disclosure. FIG. 5A depicts DNA latches such as nucleic acids-of-interest that can be placed at specific locations on the scaffold resulting in different loop sizes. FIG. 5B depicts a nanoswitch mixture with 5 different nanoswitches that can recognize specific RNA lock strands to reconfigure and yield specific bands on a gel. In embodiments, the presence or absence of these five bands can be used as a 5-bit code to encode information in DNA nanoswitches. FIG. 5C depicts a combination of different locked states of the five nanoswitches is used as a 5-bit code to encode information (e.g.: “R”, “N”, “A”), and the information can be erased by the addition of RNase H. FIG. 5D depicts using DNA locks, the specific nanoswitches can be protected from unlocking, providing a write-protection feature for information encoding. FIG. 5E is a schematic illustration depicting Information (locked bit) erased using RNase H can be re-written using a DNA lock of the same sequence as the previously used RNA lock.

FIG. 6A is a schematic illustration showing the nanoswitch is a duplex formed from linear M13 and short complementary backbone oligonucleotides. FIG. 6B depicts two DNA latches containing single-stranded overhangs that complement the RNA lock, or first nucleic acid, placed at two locations on the scaffold. FIG. 6C depicts a DNA nanoswitch-nucleic acid complex in a locked state.

FIG. 7 depicts a series of gel electrophoresis readouts showing RNase H activity is monitored with an RNA lock on the DNA nanoswitch but is inactive when a DNA lock is used.

FIG. 8 depicts a series of gel electrophoresis readouts showing optimization of RNA locking of DNA nanoswitches with different concentrations of RNA lock strand and varying incubation times.

FIG. 9 is a gel electrophoresis readout showing unlooping of DNA nanoswitches with different amounts of RNase H enzyme.

FIG. 10 is a gel electrophoresis readout showing RNase H assay at different temperatures.

FIGS. 11A-11C are schematic illustrations showing programmable loop sizes by placing DNA latches at defined positions along the scaffold DNA.

FIG. 12 is a schematic illustration of a multiplexed nanoswitch mixture yields unique outputs for specific RNA lock strands. The five unique bands can be considered as a 5-bit code for encoding information with a gel-based readout.

FIG. 13 depicts a series of gel electrophoresis readouts and plot showing a time series with different amounts of RNase H.

FIG. 14 is a flowchart of a method of reconfiguring a nucleic acid construct in accordance with the present disclosure.

### DETAILED DESCRIPTION

The nucleic acid complexes, nano-switches, compositions and methods of the present disclosure herein relate to altering the conformation of one or more DNA nanoswitches for signaling depending upon signal needs. In some embodiments, the present disclosure relates to a method of reconfiguring, reshaping or causing a conformational change to a nucleic acid, nucleic acid complex, or DNA nanoswitch. In embodiments, the methods include contacting a deoxyribonucleic acid (DNA) nanoswitch and a first nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the nucleic acid is a ribonucleic acid (RNA) and the biological specimen includes one or more ribonucleases, the first conformation changes to a second conformation characterized as open; processing the mixture under conditions sufficient to separate the first conformation, and when present, the second conformation; and reacting the first conformation, and when present, the second conformation with an indicator under conditions sufficient to form a signal. In embodiments, the first conformation is stabilized in a loop conformation by the first nucleic acid, such as an RNA. The embodiments of the present disclosure advantageously provide nucleic acid complexes such as one or more nanoswitches that provide one or more useful signals such as the presence or absence of RNase, RNase activity, or ribonuclease-operated information processing encoded by DNA. In embodiments, the present disclosure advantageously provides improved methods, compositions, and assays for the detection, or identification of one or more targets-of-interest such as one or more ribonuclease (RNase) molecules e.g., endoribonucleases producing 5′-phosphomonoesters, or species thereof.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the terms “bind” and “binding” generally refer to a non-covalent interaction between a pair of partner molecules or portions thereof that exhibit mutual affinity or binding capacity. In embodiments, binding can occur such that the partners are able to interact with each other to a substantially higher degree than with other, similar substances. This specificity can result in stable complexes that remain bound during handling steps such as chromatography, centrifugation, filtration, and other techniques typically used for separations and other processes.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (e.g., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules, and for hybridization of a DNA molecule with an RNA molecule (e.g., when a DNA nanoswitch base pairs with a target RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “elute” and “eluting” refer to the disruption of non-covalent interactions between partner molecules such that the partners become unbound from one another. In embodiments, the disruption can be effected via introduction of a competitive binding species, introduction of RNase, or via a change in environmental conditions (e.g., ionic strength, pH, or other conditions).

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

An “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be one or more segments of cDNA, genomic DNA or synthetic DNA.

The term “nucleotide” refers to a ribonucleotide or a deoxyribonucleotide or modified form thereof, as well as an analog thereof.

The term “nanoswitch” refers to a nucleic acid molecule, either single- or double-stranded, which is modified to contain segments of nucleic acids in a manner that would not otherwise exist in nature, that assumes a linear (or open) conformation in the absence of a predetermined nucleic acid or a looped (or closed) formation in the presence of the same predetermined nucleic acid.

Nucleic acid construct: The term “nucleic acid construct” as used herein refers to a nucleic acid molecule, either single- or double-stranded, which is modified to contain segments of nucleic acids in a manner that would not otherwise exist in nature.

As used herein, the term “nucleic acid molecule” refers to any molecule containing multiple nucleotides (e.g., molecules including a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (e.g., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The term “isolated” means a substance in a form or environment that does not occur in nature. Non-limiting examples of isolated substances include (1) any non-naturally occurring substance, (2) any substance such as a variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The term “polynucleotide” refers to polymers of nucleotides. In embodiments, the term polynucleotide includes but is not limited to DNA, RNA, DNA/RNA hybrids including polynucleotide chains of regularly and irregularly alternating deoxyribosyl moieties and ribosyl moieties (e.g., wherein alternate nucleotide units have an —OH, then and —H, then an —OH, then an —H, and so on at the 2′ position of a sugar moiety), and modifications of these kinds of polynucleotides wherein the attachment of various entities or moieties to the nucleotide units at any position are included.

The term “polyribonucleotide” refers to a polynucleotide including two or more modified or unmodified ribonucleotides and/or their analogs.

The term “ribonucleotide” and the phrase “ribonucleic acid” (RNA), refer to a modified or unmodified nucleotide or polynucleotide including at least one ribonucleotide unit. A ribonucleotide unit includes an oxygen attached to the 2′ position of a ribosyl moiety having a nitrogenous base attached in N-glycosidic linkage at the 1′ position of a ribosyl moiety, and a moiety that either allows for linkage to another nucleotide or precludes linkage.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (e.g., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The term “recombinant” when used herein to characterize a nucleic acid sequence such as a plasmid, vector, construct, or complex refers to an artificial combination of two otherwise separated segments of sequence, e.g., by chemical synthesis and/or by manipulation of isolated segments of nucleic acids by genetic engineering techniques.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1 (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Certain Embodiments of the Present Disclosure

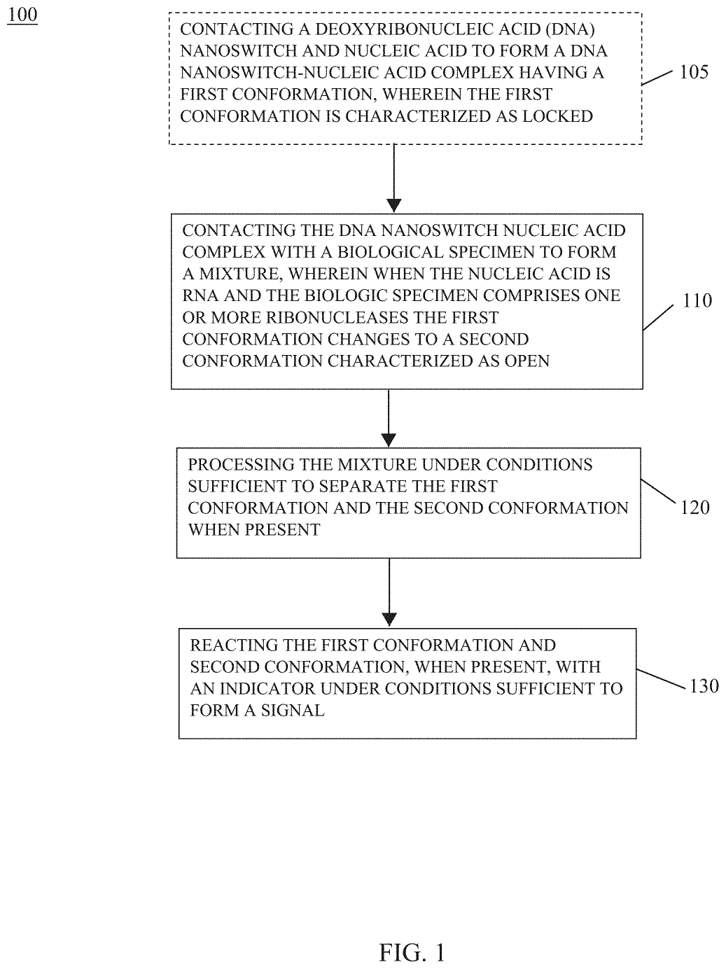

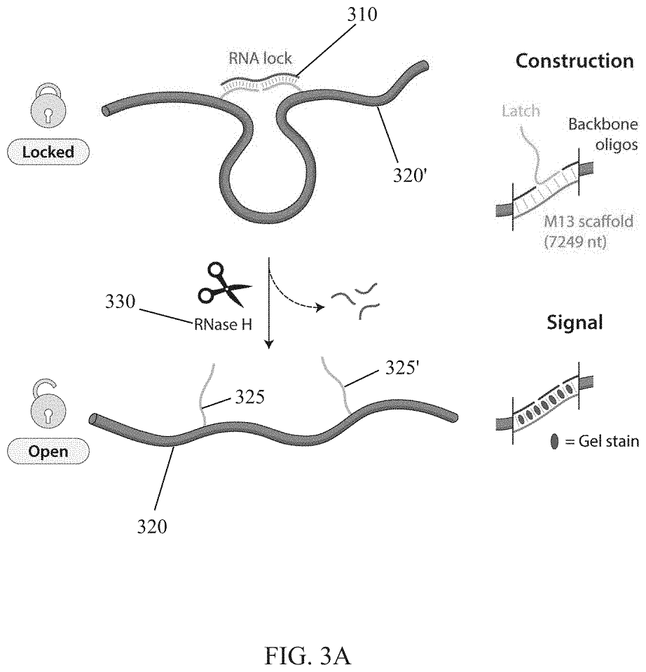

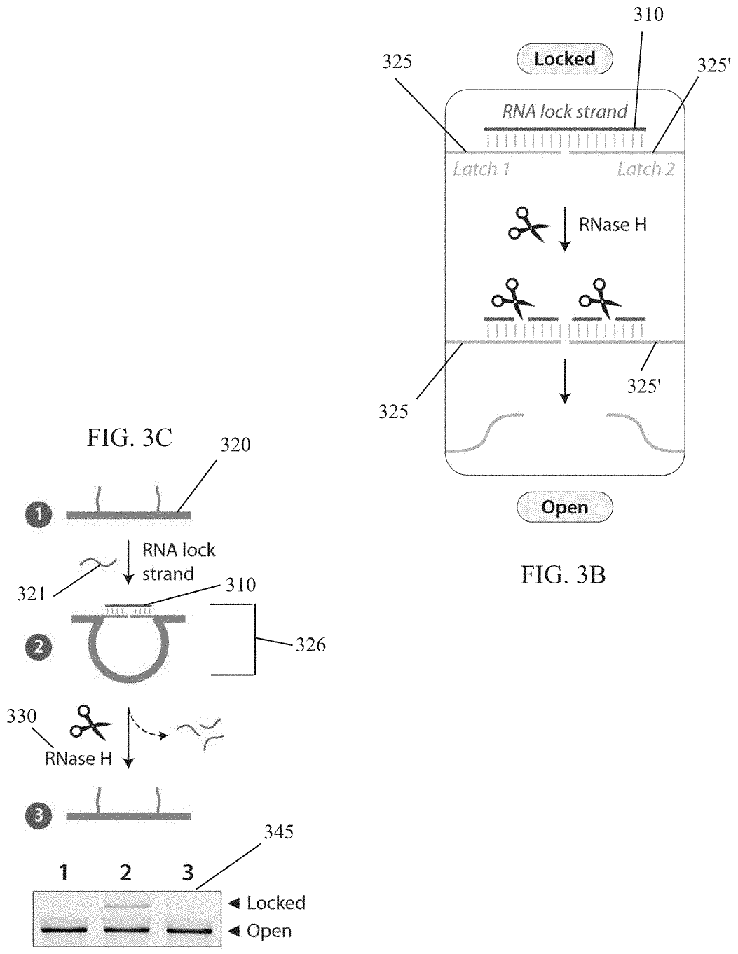

In embodiments, the present disclosure relates to one or more methods of reconfiguring a nucleic acid, polynucleotide, nucleic acid complex, or nanoswitch. In embodiments, the methods of the present disclosure include: optionally, contacting a deoxyribonucleic acid (DNA) nanoswitch and a first nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the first nucleic acid is ribonucleic acid (RNA) and the biological specimen includes one or more ribonucleases, the first conformation changes to a second conformation characterized as open; processing the mixture under conditions sufficient to separate the first conformation, and when present, the second conformation; and reacting the first conformation, and when present, the second conformation with an indicator under conditions sufficient to form a signal. FIG. 1 is a flow diagram of a method 100 for reconfiguring a nucleic acid, polynucleotide, or nanoswitch in accordance with some embodiments of the present disclosure. The method 100 is described below with respect to the stages of processing as depicted in, e.g., FIGS. 3A-3C and may be performed, for example, in a suitable labware or electrophoresis gel medium as shown below.

Initially, prior to process sequence 110, the method 100 may optionally include at process sequence 105 contacting a deoxyribonucleic acid (DNA) nanoswitch and first nucleic acid to form a DNA nanoswitch-nucleic acid complex having a first conformation, wherein the first conformation is characterized as locked. For example, where it is desirable to preselect a DNA nanoswitch for the purpose of indicating the presence of ribonuclease in a biological sample, a preselected deoxyribonucleic acid (DNA) nanoswitch may be configured to include a first nucleic acid such as a preselected RNA oligonucleotide to connect portions of the DNA nanoswitch backbone at preselected locations to form a DNA nanoswitch-nucleic acid complex having a first conformation, wherein the first conformation is characterized as locked, or has a looped or closed conformation.

In embodiments, a nucleic acid complex is provided including: a scaffold nucleic acid hybridized to a plurality of oligonucleotides, wherein a first and a second oligonucleotide in the plurality are partially hybridized to the scaffold nucleic acid, wherein the first oligonucleotide includes a 3′ overhang and the second oligonucleotide includes a 5′ overhang. In embodiments, the 3′ overhang is not complimentary to the 5′ overhang. In embodiments, if the 3′ overhang and the 5′ overhang are brought into close proximity to each other, a loop is formed in the nucleic acid complex, such as in the presence of a first nucleic acid that is complementary or substantially complementary to both the 3′ overhang and the 5′ overhang. In embodiments, the first nucleic acid is RNA. In embodiments, the 3′ and 5′ overhangs each has a length of 7 or more nucleotides. In embodiments, the 3′ and 5′ overhangs are of the same length. In embodiments, the 3′ and 5′ overhangs are of the different lengths. In embodiments, the 3′ overhang includes a 3′ hydroxyl and the 5′ overhang includes a 5′ phosphate. In embodiments, a nucleic acid complex is hybridized to a first nucleic acid that is partially complementary to the 3′ overhang and partially complementary to the 5′ overhang.

In embodiments, one or more DNA nanoswitches are preformed or preselected to combine with a preselected nucleic acid such as a first nucleic acid, polynucleotide, or oligonucleotide-of-interest or a preselected RNA or preselected DNA oligonucleotide. In embodiments, a DNA nanoswitch-nucleic acid complex suitable for use herein is a nucleic acid complex for use in detecting targets. Targets are detected based on their interactions with the DNA nanoswitch-nucleic acid complex and the conformational changes that are induced in the nanoswitches and/or DNA nanoswitch-nucleic acid complex as result of such interactions. In embodiments, the DNA nanoswitch-nucleic acid complexes are designed so that in the absence of the target they typically maintain a looped (or closed) conformation and assume a linear (or open) conformation in the presence of a target. These conformations are detected and physically separable from each other using various techniques including but not limited to gel electrophoresis. In the context of gel electrophoresis, the open and closed conformations migrate to different extents through a gel, and they can emit signal from the gel in order to, in some embodiments, further inform that the target has altered the conformation of the nanoswitch.

In embodiments, nanoswitches and DNA nanoswitch-nucleic acid complexes of the present disclosure are designed to detect a variety of targets, including but not limited to targets that are endonucleases such as one or more ribonucleases, molecules having RNase activity, and/or molecules that inhibit RNase activity. For example, in embodiments, a nanoswitch of the present disclosure is configured to bind to a binding partner that may be altered, e.g., digested, by a target-of-interest such as an endonuclease. In embodiments, the binding partner may be a preselected nucleic acid that binds to the nanoswitch and locks the nanoswitch into a closed conformation. In embodiments, the preselected nucleic acid is a nucleic acid (such as RNA, DNA, or nucleotides thereof) based on sequence complementarity. In embodiments, and as shown in FIG. 3C, nanoswitches of the present disclosure bind to a pre-selected nucleic acid and become susceptible to reacting with a target-of-interest, as the target may digest the pre-selected nucleic acid resulting in a conformational change such as a change in the conformation of the nanoswitch to an open confirmation.

Various aspects of the present disclosure relate to the use of nanoswitches to detect targets-of-interest that are endonucleases such as Rnase. In some embodiments, the target comprises or consist of RNase. In some embodiments, the RNase is one of two types of ribonucleases including endoribonucleases and exoribonucleases characterized by EC (Enzyme Commission) numbers: EC 2.7 and EC 3.1. For example, in some embodiments, the RNase is an endonuclease producing 5′-phosphomonoesters such as ribonuclease H. In some embodiments, ribonuclease H refers to a ribonuclease that cleaves the RNA in a DNA/RNA duplex to produce ssDNA. In some embodiments, RNase H refers to a non-specific endonuclease that catalyzes the cleavage of RNA via a hydrolytic mechanism, aided by an enzyme-bound divalent metal ion. In some embodiments, RNase H leaves a 5′-phosphorylated product. In some embodiments, RNase includes endonucleases from Enzyme Class EC 3.1.27.5 or Rnase A. in embodiments, RNase A refers to endonucleases producing other than 5′-phosphomonoesters.

In embodiments, detection of one or more targets-of-interest such as RNase is important for a variety of applications including for example in the fields of medicine and forensics. In some embodiments, the present disclosure provides a programmable nucleic acid-based nanoswitch that undergoes a pre-defined conformational change upon contact with a target nucleic acid such as target RNase, converting a nanoswitch within a DNA-nanoswitch-nucleic acid complex from a looped or “locked” state to a linear or “open” state (or conformation).

In embodiments, the looped “locked” state relates to a DNA complex or conformation that includes a combination of the DNA nanoswitch and a preselected nucleic acid combined to form a DNA nanoswitch-nucleic acid complex having a first conformation, wherein the first conformation is characterized as locked. In embodiments, the nucleic acid is selected to react or not react when contacted with a target-of-interest. For example, when the preselected nucleic acid is a ribonucleic acid oligonucleotide, the ribonucleic acid oligonucleotide may be preselected to digest when contacted with a target-of-interest such an RNase or molecule having RNase activity.

In embodiments, a DNA nanoswitch and/or DNA nanoswitch nucleic acid complex can be detected using separation techniques such as standard gel electrophoresis, which are capable of physically separating the open and locked conformations from each other and from other components in a mixture, and in some instances also are capable of facilitating isolation of DNA nanoswitch nucleic acid complex having a first conformation and/or nanoswitch having a second conformation different than the first conformation. In embodiments, other separation medium suitable for use herein may include liquid chromatography medium such as those used HPLC columns, or other medium such as those used in capillary electrophoresis.

In embodiments, the present disclosure demonstrates successful detection of one or more RNases or molecules having RNase activity. The detection method can be accomplished quickly, including as demonstrated herein within 15 minutes from sample mixture to readout. The approach is a low cost and technically accessible, and thus well-suited for point-of-use detection.

In addition, the compositions of the present disclosure may also be used to simultaneously detect more than one target-of-interest. For example, the nanoswitch nucleic acid complex may be designed to include one or more nucleic acids configured to form the close looped shaped, wherein the one on more nucleic acids have different lengths and/or discernable structure. Variation in loop size may facilitate varying reaction conditions among a plurality of targets-of-interest such as different types of RNase and facilitate detection between various RNases.

In embodiments, a nucleic acid construct such as a DNA nanoswitch and/or DNA nanoswitch-nucleic acid complex, as described herein includes a scaffold nucleic acid hybridized in a sequence specific manner to a plurality of oligonucleotides. The scaffold and the oligonucleotides may be referred to herein as being single-stranded. In embodiments, prior to hybridization to each other, both nucleic acid species are single-stranded. In embodiments, upon hybridization, a double-stranded nucleic acid is formed. Typically, the oligonucleotides hybridize to the scaffold nucleic acid in a consecutive, non-overlapping, manner.

In some non-limiting embodiments, the nucleic acid constructs such as nanoswitches are formed by hybridizing a scaffold nucleic acid to one or more oligonucleotides. The disclosure contemplates any variety of means and methods for generating the nanoswitches and nanoswitch-nucleic acid complexes described herein. It is also to be understood that while for the sake of brevity the disclosure refers to oligonucleotides that are hybridized to a scaffold nucleic acid, such a complex may have been formed by hybridizing single stranded scaffold to single stranded oligonucleotides, but it is not intended that it was exclusively formed in this manner. In embodiments, the nucleic acid complexes such as nanoswitches and nanoswitch-nucleic acid constructs may include double-stranded and single-stranded regions. As used herein, a double-stranded region is a region in which all nucleotides on a scaffold are hybridized to their complementary nucleotides on the oligonucleotide. Double-stranded regions may include “single-stranded nicks” as the hybridized oligonucleotides typically are not ligated to each other. The single-stranded regions are scaffold sequences that are not hybridized to oligonucleotides. Certain complexes may include one or more single-stranded regions in between double-stranded regions (typically as a result of unhybridized nucleotides in between adjacent hybridized oligonucleotides). The complexes may be at least 50%, at least 60%, at least 70%, at least 80%, at least 90%, at least 95%, at least 96%, at least 97%, at least 98%, at least 99%, or 100% double-stranded. In some embodiments, they are at least 80% double stranded.

In embodiments, the DNA nanoswitch are modular complexes to which can be attached one or more nucleic acids of interest such as one or more oligonucleotides, one or more binding pairs of interest, nucleic acids, and the like. The terms attach, link and conjugate are used interchangeably throughout this disclosure unless otherwise stated. In embodiments, the combination of a DNA nanoswitch and nucleic acid, or nucleic acid-of-interest forms a DNA nanoswitch-nucleic acid complex of the present disclosure, in a locked or looped conformation. In embodiments, removal of the nucleic acid, or nucleic acid-of-interest forms a DNA nanoswitch of the present disclosure, in an open, non-looped, or linear conformation.

In embodiments, the nanoswitches of the present disclosure provided herein are stable in complex fluids such as but not limited to serum-containing samples, including up to 30% FBS. In some embodiments, nanoswitches for use herein are configured to convert from unbound to bound forms in the presence of complex fluids (e.g., 30% FBS). Moreover, the nanoswitches are also stable for an extended period of time. Once synthesized, the nanoswitches may be dried and stored for days, weeks or months. Similarly, DNA nanoswitch-nucleic acid complexes of the present disclosure may be stable for an extended period of time. Once synthesized, the DNA nanoswitch-nucleic acid complexes may be dried and stored for days, weeks or months.

In some embodiments, the nanoswitches of the present disclosure and/or DNA nanoswitch-nucleic acid complexes of the present disclosure can be made using nucleic acid nanostructure techniques such as but not limited to DNA origami. (Rothemund P. W. K. (2006) Nature 440: 297-302; Douglas S. M. et al. (2009) Nature 459: 414-8). In embodiments, the nanoswitches of the present disclosure may be formed as described in U.S. Patent Publication No. 2018/0223344 entitled Compositions and Methods for Analyte Detection Using Nanoswitches published on 9 Aug. 2018 to Chandrasekaren et al. (herein entirely incorporated by reference). In embodiments, one or more nanoswitches of the present disclosure include an RNA lock segment of RNA as shown in FIG. 3A. In embodiments, the combination of a DNA nanoswitch and nucleic acid (such as the RNA lock segment) forms a DNA nanoswitch-nucleic acid complex of the present disclosure, in a locked or looped conformation. In embodiments, the combination of a DNA nanoswitch and nucleic acid (such as the RNA lock segment) forms a stable DNA nanoswitch-nucleic acid complex of the present disclosure, in a locked or looped conformation.

### Scaffolds

In embodiments, scaffold nucleic acid suitable for use herein may be of any length sufficient to allow association (e.g., binding) and dissociation (e.g., unbinding) of binding partners to occur and to be distinguished from other association and/or dissociation events using the read out methods provided herein, including gel electrophoresis.

In embodiments, the scaffold nucleic acid is at least 500 nucleotides in length, and it may be as long as 50,000 nucleotides in length (or it may be longer). The scaffold nucleic acid may therefore be 1000-20,000 nucleotides in length, 1000-15,000 nucleotides in length, 1000-10,000 in length, or any range therebetween. In some embodiments, the scaffold ranges in length from about 5,000-10,000 nucleotides, and may be about 7000-7500 nucleotides in length or about 7250 nucleotides in length.

In some embodiments, the scaffold may be a naturally occurring nucleic acid (e.g., M13 scaffolds such as M13mp18). M13 scaffolds are disclosed by Rothemund 2006 Nature 440:297-302, the teachings of which are incorporated by reference herein. Such scaffolds are about 7249 nucleotides in length.

In some embodiments, the scaffold nucleic acid may also be non-naturally occurring nucleic acids such as polymerase chain reaction (PCR)-generated nucleic acids, rolling circle amplification (RCA)-generated nucleic acids, etc. In some embodiments, the scaffold nucleic acid is rendered single-stranded either during or post synthesis. Methods for generating a single-stranded scaffold include asymmetric PCR. Alternatively, double-stranded nucleic acids may be subjected to strand separation techniques in order to obtain the single-stranded scaffold nucleic acids. The scaffold nucleic acid may comprise DNA, RNA, DNA analogs, RNA analogs, or a combination thereof, provided it is able to hybridize in a sequence-specific and non-overlapping manner to the oligonucleotides. In some instances, the scaffold nucleic acid is a DNA.

### Oligonucleotides

In embodiments, the scaffold nucleic acid is hybridized to a plurality of oligonucleotides. In embodiments, each of the plurality of oligonucleotides is able to hybridize to the scaffold nucleic acid in a sequence-specific and non-overlapping manner (i.e., each oligonucleotide hybridizes to a distinct sequence in the scaffold). The length and the number of oligonucleotides used may vary. In some instances, the length and sequence of the oligonucleotides is chosen so that each oligonucleotide is bound to the scaffold nucleic acid at a similar strength. This is important if a single condition is used to hybridize a plurality of oligonucleotides to the scaffold nucleic acid, such as for example in a one-pot synthesis scheme.

In embodiments, the number of oligonucleotides will depend in part on the application, the length of the scaffold, and the length of the oligonucleotides themselves. In embodiments, the oligonucleotides are designed to be of approximately equal length. In some embodiments, the oligonucleotides may be about 20-100 nucleotides in length. The oligonucleotides may be, without limitation, about 20, about 30, about 40, about 50, about 60, about 70, about 80, about 90 or about 100 nucleotides in length. In some embodiments, the oligonucleotides may be about 40-80 nucleotides in length. In some embodiments, the oligonucleotides may be about 60 nucleotides in length.

The number of oligonucleotides in the plurality may be about 70, about 80, about 90, about 100, about 110, about 120, about 130, about 140, about 150, about 160, about 170, about 180, about 190, about 200, about 300, about 400, about 400, about 500, about 600, about 700, about 800, about 900, or about 1000, without limitation.

In some embodiments, the nucleic acid complex may include the M13 ssDNA as the scaffold and about 120 oligonucleotides each equal to or about 60 nucleotides in length.

In embodiments, the oligonucleotides may be characterized as modified or unmodified or variable oligonucleotides. In embodiments, the variable oligonucleotides may be conjugated to reactive groups that are not normally present in a nucleic acid sequence, such as for example click chemistry reactive groups, or they may be conjugated to target-specific binding partners such as antibodies or antibody fragments, or they may include other moieties which are not typically present in an unmodified oligonucleotide. An example is a variable oligonucleotide including a phosphate at their 5′ end (referred to herein as a 5′ phosphate). Oligonucleotides having this latter modification are used herein in the detection of target nucleic acids, and in this context such oligonucleotides are referred to as “detector” strands since they are preselected to bind to nucleic acids of the interest via hybridization to form a DNA Nanoswitch-nucleic acid complex having a first conformation, wherein the first conformation is characterized as locked.

In some instances, the first and last oligonucleotides as well as “internal” oligonucleotides, typically at pre-defined positions along the length of the scaffold, may be modified oligonucleotides. The position of the variable oligonucleotides may be, but are not necessarily, evenly distributed along the length of the scaffold.

### Binding Interactions and Looped Conformations

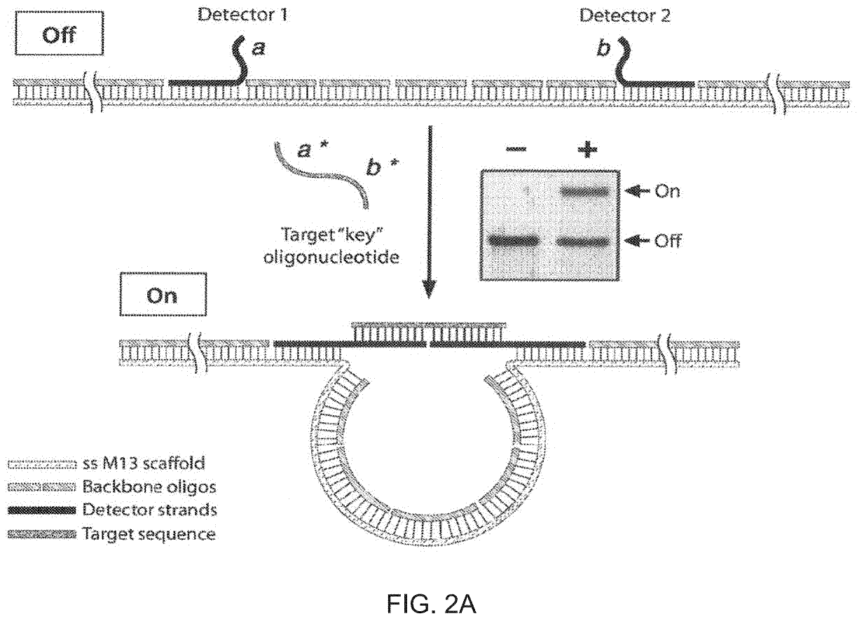

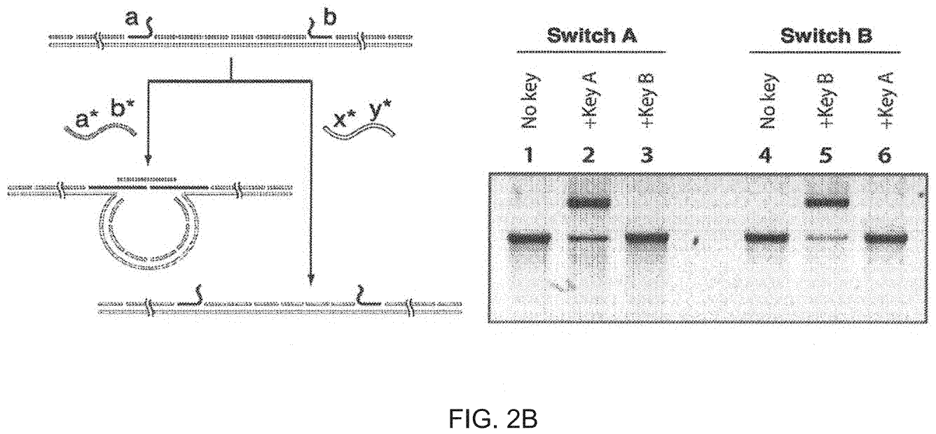

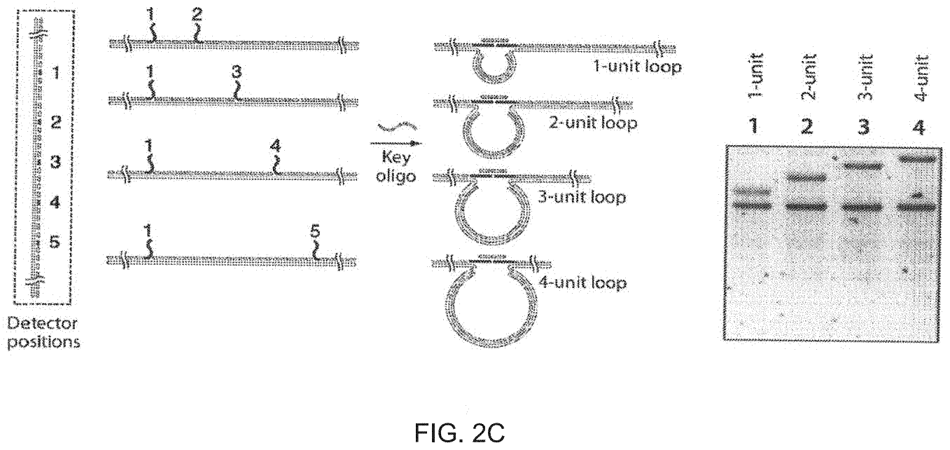

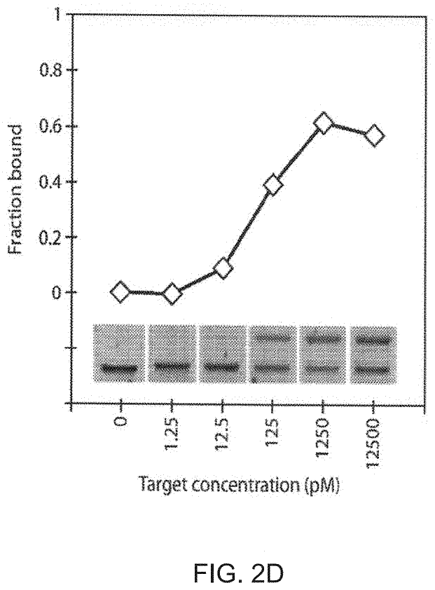

In embodiments, the location of the variable oligonucleotides dictates the location of the various substituents in the complex, such as detector strands, nucleic acid binding partners, etc. It also dictates the size of the loops that are formed once the various substituents bind to each other, as shown in FIGS. 2A and 2C. This will in turn dictate the migration distance of the looped (closed) complex, and thus the ability of the end user to physically separate and thus distinguish between complexes of interest (e.g., locked complexes such as nanoswitch-nucleic acid complexes having a first conformation, wherein the first conformation is characterized as locked and/or looped) and nanoswitch-nucleic acid complexes having a second conformation (e.g., open complexes such as where the nucleic acid-of-interest is digested and the nanoswitch has a non-looped or linear conformation).

In embodiments, a nanoswitch may include a first and a second oligonucleotide such as a first and second detector strand that together hybridize to one or more preselected nucleic acids-of-interest such as preselected ribonucleic acid or pre-selected deoxyribonucleic acid. In these embodiments, the hybridization of the nanoswitch to the nucleic acid is considered a first binding interaction. Alternatively, a second binding interaction may be an additional binding interaction that occurs upon hybridization of a second nucleic acid such as to a second pair of detector strands.

In embodiments, the nanoswitch is designed to detect one target ribonuclease by hybridization of a first oligonucleotide and a second oligonucleotide, each having an overhang (e.g., a single-stranded region that is available for hybridization to the nucleic acid such as an oligo-ribonucleotide). Such overhangs are shown in FIG. 2A. The first and second oligonucleotides, in this example, may be referred to as partially hybridized to the scaffold since each has a single-stranded overhang region and a region that is hybridized to the scaffold. The first and second oligonucleotides are denoted “detector 1” and “detector 2”. The overhangs may be referred to herein as 3′ overhangs and 5′ overhangs, referring to the directionality of the single-stranded region. The distance between the first and the second oligonucleotides, when bound to the scaffold, dictates the size of the loop and ultimately the migration distance of the nanoswitch when it is bound to the target (or when it is stabilized) via a latch binding interaction. In embodiments, the detector length may have a length of 5 to 30 or 7-20 nucleotides.

In some embodiments, the first oligonucleotide and the second oligonucleotide are separated from each other by 100-6000 nucleotides. In some instances, the first oligonucleotide and the second oligonucleotide are separated from each other by 500 to 5000 nucleotides, 600-5000 nucleotides, 1000-5000 nucleotides, or 1000-3000 nucleotides. In some embodiments, the first oligonucleotide and the second oligonucleotide are separated from each other by at least 500, 600, 700, 800, 900, 1000, 1500, 2000, 2500, 3000, 3500, 4000, 4500, 5000, 5500, 6000, or more nucleotides. In some embodiments, the first oligonucleotide and the second oligonucleotide are located about equidistant about the center of the scaffold nucleic acid. In some embodiments, the first and second oligonucleotides bind to regions of the scaffold nucleic acid that are internal to the scaffold (i.e., such regions exclude the most 5′ and the most 3′ nucleotides of the scaffold).

### Gel Electrophoresis

In embodiments, such as when measured using gel electrophoresis, the open and closed nanoswitch conformations migrate differentially through a medium such as a gel medium. In embodiments, a circular scaffold such as circular M13 migrates the slowest, a linearized double-stranded version of M13 (without internal binding interactions) migrates fastest, and nanoswitches in looped conformations migrate in between. In embodiments, the migration distance differs based on the length of the loop. As an example, loops that are on the order of about 2590 base pairs are clearly distinguishable from loops that are on the order of about 600 base pairs. Loops of other sizes can also be distinguished from each other, as described herein, and as demonstrated for example in FIG. 2C. The ability to distinguish between loops of different sizes means that the presence (or absence) of multiple targets (each detected by a complex having a loop of a particular size) can be determined simultaneously in a multiplexed assay. Such methods may be used to detect the presence of a single or multiple target and may form the basis of a diagnostic assay. Moreover, it should also be understood that nanoswitches having one loop can also be distinguished from nanoswitches having more than one loop, including those that have 2, 3 or more loops. In embodiments, a single type of nanoswitch can be used to detect two different targets and depending on the conformation of the nanoswitch (as determined by its migration distance in a gel), an end user can determine whether either or both targets are present in a sample. These nanoswitches can then also be extracted from the gel and the bound targets can be isolated.

In embodiments, electrophoresis is performed wherein a gel is run at 4 degrees Celsius to maintain the interaction of the targets to their binding partners (e.g., the binding of a protein target to target-specific antibodies) or to maintain latch binding interactions. It is contemplated that other separation medium is suitable for use herein such those used in capillary electrophoresis and liquid chromatography.

### Nanoswitches

In some embodiments, nanoswitches designed for DNA nanoswitch-nucleic acid complex formation are provided. In some embodiments, nanoswitches of the present disclosure include a scaffold nucleic acid hybridized to a plurality of oligonucleotides such as detectors, as described herein. A portion of an exemplary nanoswitch is provided in FIG. 2A. As illustrated, the nanoswitch includes a first and a second oligonucleotide that are partially hybridized to the scaffold nucleic acid (i.e., each of these oligonucleotides is partially hybridized to the scaffold and thus each is partially single-stranded). The first oligonucleotide includes a 3′ overhang and the second oligonucleotide includes a 5′ overhang.

In embodiments, the 3′ overhang is not complementary to the 5′ overhang, and rather both the 3′ and the 5′ overhangs are complementary to a nucleic acid-of-interest suitable for forming a DNA nanoswitch-nucleic acid complex of the present disclosure. FIG. 2A illustrates an embodiment in which the entire nucleic acid-of-interest (referred to in the Figure as “Target “Key” oligonucleotide”) hybridizes to a combination of the 3′ and 5′ overhang. However, in embodiments, the method can also be performed in which the 3′ and 5′ overhangs are designed to hybridize only the 5′ and 3′ regions of a nucleic acid-of-interest, with the internal or middle region of the target nucleic acid remaining unhybridized. In this latter instance, the nanoswitch is designed to include a plurality of nucleic acids-of-interest differing sequences provided that they are at least complementary to the 3′ and 5′ overhangs. In embodiments, the nanoswitch detects non-adjacent sequences on the target. Such non-adjacent sequences may be separated by 1 or 2 nucleotides or by 10's or 100's of nucleotides, without limitation. As shown in FIG. 3A shows a nucleic acid such as nucleic acid-of-interest 310 hybridized to the nanoswitch 320. In embodiments, the nucleic acid such as nucleic acid-of-interest 310 is a binding partner that stabilizes the nanoswitch in a looped conformation. In embodiments, the removal or digestion of the nucleic acid such as nucleic acid-of-interest 310 will destabilize a looped conformation, and the complex will undergo a conformation change to a linear conformation as shown in FIG. 3C.

Referring to FIGS. 3A-3C, a nucleic acid, or polynucleotide of the present disclosure is shown including a DNA nanoswitch-nucleic acid complex including a deoxyribonucleic acid (DNA) nanoswitch and an oligonucleotide, wherein the DNA nanoswitch and oligonucleotide form, or are configured to form, a DNA-nanoswitch-nucleic acid complex having a first conformation characterized as locked or looped, and a second conformation characterized as open or linear when in a presence of ribonuclease. In embodiments, the oligonucleotide is a nucleic acid-of-interest as described herein. In embodiments, the oligonucleotide is a single stranded ribonucleic acid.

In embodiments, the nanoswitch is configured such that the 3′ and 5′ overhangs come into sufficient proximity to each other in the presence of the nucleic acid-of-interest, and that it is only once the nucleic acid-of-interest hybridizes to the 3′ and 5′ overhangs that a looped conformation of a DNA-nanoswitch-nucleic acid complex is formed.

In some embodiments, the overhangs may be of different or identical lengths, relative to each other. The overhang length may range from 5-20 nucleotides in length, without limitation. The overhangs may have a length of 5 or more, or 6 or more, or 7 or more nucleotides. One or both overhangs may have a length of 7, 8, 9, 10, 11, 12, 13, 14, 15, 16, 17, 18, 19 or 20 nucleotides. The combined length of the overhangs may vary and may depend on their sequence and the length of the target nucleic acid. Their combined length may be 14 nucleotides or longer, without limitation. In some instances, the 3′ overhang and the 5′ overhang are of different lengths and their combined length is at least about 22 nucleotides.

In some embodiments, the combined length of the overhangs may be the same length as the nucleic acid-of-interest. Alternatively, the combined length of the overhangs may be longer or shorter than the length of the nucleic acid-of-interest. In some embodiments, the nucleic acid-of-interest may not bind to both overhangs to the same extent. In other words, one overhang may share more sequence complementarity with the nucleic acid-of-interest than the other overhang. In some embodiments, the overhangs will be referred to herein as the 3′ and 5′ overhangs intending the directionality of the overhangs. In some embodiments, the overhangs will be ligated to each other, as described herein, and thus the 3′ overhang may comprise a 3′ hydroxyl and the 5′ overhang may comprise a 5′ phosphate.

In some embodiments, the overhangs may be designed such that they include secondary structure such as but not limited to hairpin conformations. Such secondary structures may be melted during hybridization to the target, or they may be melted as a result of a change in condition or contact with an extrinsic trigger. Thus, also provided herein are compositions comprising any of the foregoing nucleic acid complexes. The composition may comprise a plurality of nucleic acid complexes. The nucleic acid complexes in the plurality may be identical to each other.

Alternatively, the nucleic acid complexes in the plurality may be different from each other. The nanoswitches may differ from each other with respect to their nucleic acid-of-interest specificity (e.g., the nucleotide sequence of their 3′ overhangs and/or the sequence of the 5′ overhangs). Nanoswitches may also differ from each other with respect to the distance between the 3′ overhang and the 5′ overhang along the length of the scaffold nucleic acid.

Referring now to FIG. 2B a schematic illustration shows the sequence specificity of embodiments of one or more DNA nanoswitches in accordance with the present disclosure. For example, an agarose gel shows the sequence specificity of the nanoswitch. Switch A turns on only in the presence of key oligonucleotide A and switch B turns on only in the presence of key oligonucleotide B with no background detection of the incorrect strand.

### Nucleic Acid-of-Interest

In embodiments, and as described above, one or more nucleic acids such a first nucleic acid, or nucleic acids-of-interest may be pre-selected to hybridize to the one or more detectors and form a DNA-nanoswitch-nucleic acid complex of the present disclosure. In the embodiments, the one or more nucleic acids-of-interest may be a DNA, RNA or a combination thereof. In embodiments, the nucleic acid-of-interest may be a naturally occurring nucleic acid, recombinantly added to the DNA nanoswitch of the present disclosure. Examples include an miRNA, a tumor-specific nucleic acid, an allelic variant, and the like, without limitation.

In embodiments, the nucleic acid or nucleic acid-of-interest, as used herein, refers to the nucleic acid that is hybridized to the nanoswitch. It is to be understood that the nucleic acid-of-interest may derive from and thus be a fragment of a much larger nucleic acid such as for example genomic DNA or an mRNA. Thus, a binding portion of the nucleic acid-of-interest (i.e., the nucleic acid bound to the nanoswitch) may range from about 7-50 nucleotides, or e.g., 10 to 35 nucleotides, or 5 to 30 nucleotides in some instances, while its parent nucleic acid may be much longer (for example on the order to kbs or more).

In some embodiments, the conditions that allow nucleic acid-of-interest such as RNA to hybridize to the 3′ overhang and the 5′ overhang may be standard hybridization conditions as known in the art. Such conditions may include a suitable concentration of salt(s) and optionally a buffer. The condition may also include EDTA in order to preserve the DNA nanoswitch-nucleic acid complex.

In some embodiments, the hybridization may be accomplished using a constant annealing temperature. Such constant temperature may range from about 4° C. to 55° C., 15° C. to 30° C., or 20° C. to 30° C., or may be about 25° C. The temperature may be regarded as room temperature (RT). The hybridization may be carried out over a period of hours such as 1, 2, 3, 4, 5 hours or more.

In some embodiments, the hybridization may be accomplished by decreasing the temperature from a temperature at which the nucleic acid-of-interest and the overhangs are not hybridized to each other to a temperature at which they are hybridized to each other. This is referred to herein as a temperature ramp or a decreasing annealing temperature. The starting temperature may be about 40-60° C. without limitation. The ending temperature may be about 4-25° C. without limitation. Thus, the temperature ramp may be from about 50° C. to about 4° C. or about 40° C. to about 4° C. In embodiments, a temperature ramp from about 46° C. to about 4° C. The change in temperature is typically carried out over 1-12 hours. Thus, the change in temperature may decrease by about 0.1-1° C. per minute.

Regardless of whether a constant or decreasing annealing temperature is used, the hybridization may also be carried out for much shorter periods of time, for example on the order of 10-30 minutes, provided readout can be achieved. Thus, in some instances, if the method determines if the nucleic acid-of-interest is present and has formed a DNA nanoswitch-nucleic acid complex of the present disclosure, then the hybridization period can be short, particularly if the nucleic acid-of-interest is present in abundance. In some embodiments, longer hybridization times may be required. Similarly, if the nucleic acid-of-interest is present in low abundance, longer hybridization times may be required, particularly if an amplifying latch mechanism is used.

In some embodiments, only a portion or preselected portion of the nucleic acid-of-interest hybridizes to the nanoswitch of the present disclosure.

### Test Sample(s)

In embodiments, the biological sample is a sample that is being tested for the presence of one or more targets-of-interest such as an endonuclease as described above, molecules having RNase activity, and/or molecules that inhibit RNase activity.

In embodiments, a target-of-interest may be present and thus contacted with a DNA nanoswitch-nucleic acid complex of the present disclosure within in a mixture such as a biological sample or specimen. In embodiments, a target-of-interest may be disposed within a sample that may contain the target(s) or it may be suspected of containing the target(s). In embodiments, a sample may comprise non-target nucleic acid and be in the form of a mixture. Non-target nucleic acid, as used herein, refers to nucleic acids that are not the targets-of-interest or do not include a binding portion to the nanoswitch, or do not interact with the nanoswitch in a manner that will cause a conformational change. In embodiments, the non-targets may be a peptide, protein or the like that does not react with or alter the conformation of the DNA nanoswitch-nucleic acid complex. The methods provided herein allow for the detection of a target even if such target is present in a molar excess of non-target nucleic acid. Thus, the sample may comprise on the order of micromolar quantities of non-target nucleic acid or protein and only nanomolar or picomolar quantities of target and still be able to detect the target. The target and non-targets may be present in the sample ata molar ratio of 1:10², 1:10³, 1:10⁴, 1:10⁵, or up to 1:10⁹.

In some embodiments, the sample may be or may derived from a biological sample such as a bodily fluid (e.g., a blood sample, a urine sample, a sputum sample, a stool sample, a biopsy, and the like). The disclosure contemplates that such samples may be manipulated prior to contact or mixing with the nanoswitches or DNA nanoswitch-nucleic acid complexes of the present disclosure. For example, the samples may be treated to lyse cells, degrade or remove protein components, fragment nucleic acids such as genomic DNA, and the like.

In some instances, the target is or is derived from or is a fragment of an endonuclease. In some embodiments the target is RNase such as RNase H, or RNase A. In embodiments, the target is able to interact with and/or destabilize the nucleic acid-of-interest.

In embodiments, the methods of the present disclosure include detection of endonuclease. Such methods may be used to diagnose a condition, and thus may be referred to herein as diagnostic methods.

In embodiments, a method of the present disclosure includes contacting any of the foregoing DNA nanoswitch-nucleic acid complexes with an endonuclease sample under conditions that allow a target, if present in the sample, to remove or digest (partially or wholly) the nucleic acid-of-interest within the DNA nanoswitch-nucleic acid complex, to form a DNA nanoswitch without the nucleic acid-of-interest. In embodiments, the methods include detecting a conformation change between a DNA nanoswitch-nucleic acid complex to a DNA nanoswitch by moving the constructs through a separation medium such as a gel medium. In embodiments, wherein a looped conformation is present within a medium such as an electrophoresis gel medium, the conformation is indicative of the absence of a specific target-of-interest such as a target endonuclease (e.g., RNase) in the biological sample. In embodiments, in the presence of a target endonuclease, the DNA-nanoswitch-nucleic acid complex adopts a linear conformation as the DNA nanoswitch-target complex loses the nucleic acid-of-interest by the digestion, and an linear, or open conformation is formed.

In some embodiments, the conformation of the nucleic acid complex, e.g., nanoswitch may be determined (or detected) using gel electrophoresis or liquid chromatography, or other separation technique. The gel electrophoresis may be a bufferless gel electrophoresis such as the E-Gel®. Agarose Gel Electrophoresis System (Life Technologies). In embodiments, methods may include detection of the target nucleic acid and detection and optionally purification or substantial purification of the target nucleic acid. In some embodiments, the method may also include measuring an absolute or relative amount of target nucleic acid. This can be done for example by measuring the intensity of bands on a gel or of fractions from a liquid chromatography separation.

Referring now to FIG. 2D, data is providing relating to a limit of detection of nucleic acid of interest. FIG. 2D shows the signal of the on-state at different target DNA concentrations. The gel of the looped and unlooped nanoswitches is shown as the inset.

In embodiments, the nanoswitches are robust, yielding reproducible results at a variety of DNA and RNA nucleic acid-of-interest concentrations including but not limited to 0.25 nM up to 25 nM.

Referring back to FIG. 1, method 100 may start at process sequence 110 by contacting the DNA nanoswitch-nucleic acid complex with a biological specimen to form a mixture, wherein when the nucleic acid is ribonucleic acid (RNA) and the biological specimen include one or more ribonucleases, the first conformation changes to a second conformation characterized as open. In embodiments, the DNA nanoswitch-nucleic acid complex includes a DNA nanoswitch component and a nucleic acid-of-interest component hybridized to the DNA nanoswitch as described above. For example, the DNA nanoswitch-nucleic acid complex includes a DNA nanoswitch component and a nucleic acid-of-interest component hybridized to the DNA nanoswitch in the form of a first conformation wherein the DNA nanoswitch-nucleic acid target complex is in a closed loop configuration, e.g., as generally shown in FIG. 3A. In embodiments, DNA nanoswitch-nucleic acid complex can be used to determine the presence of one or more targets-of-interest such as but not limited to target endonucleases or target proteins from a sample. The looped conformation DNA nanoswitch-nucleic acid target complex can be physically separated using gel electrophoresis from linear conformation nanoswitches, which are formed when the target-of-interest is in the sample, and a mixture is formed under conditions sufficient for the target-of-interest to interact with the DNA nanoswitch-nucleic acid complex. The looped conformation DNA nanoswitch-nucleic acid complex therefore may be physically separated from a complex mixture. In embodiments, the non-looped DNA nanoswitch can also be separated from the sample or mixture.

Referring now to FIG. 1 once a DNA nanoswitch-target complex (e.g. looped conformation DNA nanoswitch-target complex are contacted with a biological specimen to form a mixture medium, wherein when the nucleic acid is ribonucleic acid (RNA) and the biological specimen comprises one or more ribonucleases, the first conformation changes to a second conformation characterized as open, process sequence 120 includes processing the mixture under conditions sufficient to separate the first conformation, and when present, the second conformation. For example, in embodiments, the locked and open conformations can be separated using gel electrophoresis. In some embodiments, specific RNA nucleic acids-of-interest will be present in the looped conformation nanoswitches, and these looped conformation nanoswitches, e.g., a DNA nanoswitch-target complex can be isolated by gel extraction from electrophoresis forming a DNA nanoswitch-target complex within a gel medium.

Referring now to FIG. 1 once processing the mixture under conditions sufficient to separate the first conformation, and when present, the second conformation is complete, process sequence 130 includes reacting the first conformation, and when present, the second conformation with an indicator under conditions sufficient to form a signal. In embodiments, the reacting may include contacting the separation medium with a dye. In some embodiments, reacting may be performed during or after gel electrophoresis, a visible signal within the gel medium.

In some embodiments, the present disclosure relates to a method of reconfiguring a nucleic acid construct including: contacting a deoxyribonucleic acid (DNA) nanoswitch and nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the nucleic acid is ribonucleic acid (RNA) and the biological specimen comprises one or more ribonucleases, the first conformation changes to a second conformation characterized as open; processing the mixture under conditions sufficient to separate the first conformation, and when present, the second conformation; and reacting the first conformation, and when present, the second conformation with an indicator under conditions sufficient to form a signal. In some embodiments, the nucleic acid is deoxyribonucleic acid or ribonucleic acid. In some embodiments, the nucleic acid is characterized as an oligonucleotide or polynucleotide having a preselected length. In some embodiments, the nucleic acid binds to the deoxyribonucleic acid (DNA) nanoswitch to form a first conformation comprising a loop. In some embodiments, the loop has a preselected size. In some embodiments, the biological specimen includes one or more ribonucleases. In some embodiments, the one or more ribonucleases comprises an endonuclease capable of producing one or more 5′ phophomonoesters. In some embodiments, the endonuclease is ribonuclease H. In some embodiments, processing the mixture includes electrophoresing the mixture under conditions sufficient to separate the first conformation and the second conformation. In some embodiments, the nucleic acid is deoxyribonucleic acid (DNA), and wherein the nucleic acid binds to the deoxyribonucleic acid (DNA) nanoswitch to form a first conformation including a loop, wherein the loop is characterized as unchanging in a presence of one or more ribonucleases. In some embodiments, a formation of the second conformation signals a presence of one or more ribonucleases (RNases). In some embodiments, the second conformation has an altered functionality compared to the first conformation. In some embodiments, the nucleic acid is an RNA oligonucleotide configured to hold the deoxyribonucleic acid (DNA) nanoswitch in the first conformation, and wherein the RNA oligonucleotide is configured to provide a code. In some embodiments, the nucleic acid is an RNA oligonucleotide configured to hold the deoxyribonucleic acid (DNA) nanoswitch in the first conformation, and wherein the RNA oligonucleotide is configured to provide a code. In some embodiments, the nucleic acid is a DNA oligonucleotide configured to hold the deoxyribonucleic acid (DNA) nanoswitch in the first conformation, and wherein the DNA oligonucleotide is configured to maintain the first conformation in a presence of ribonuclease. In some embodiments, the first conformation is configured to change to a second conformation when contacted with target-of-interest, and the second conformation is configured to report a target-of-interest. In some embodiments, the nucleic acid is an oligonucleotide configured to hold the deoxyribonucleic acid (DNA) nanoswitch in the first conformation, and wherein the oligonucleotide is configured to provide a code.

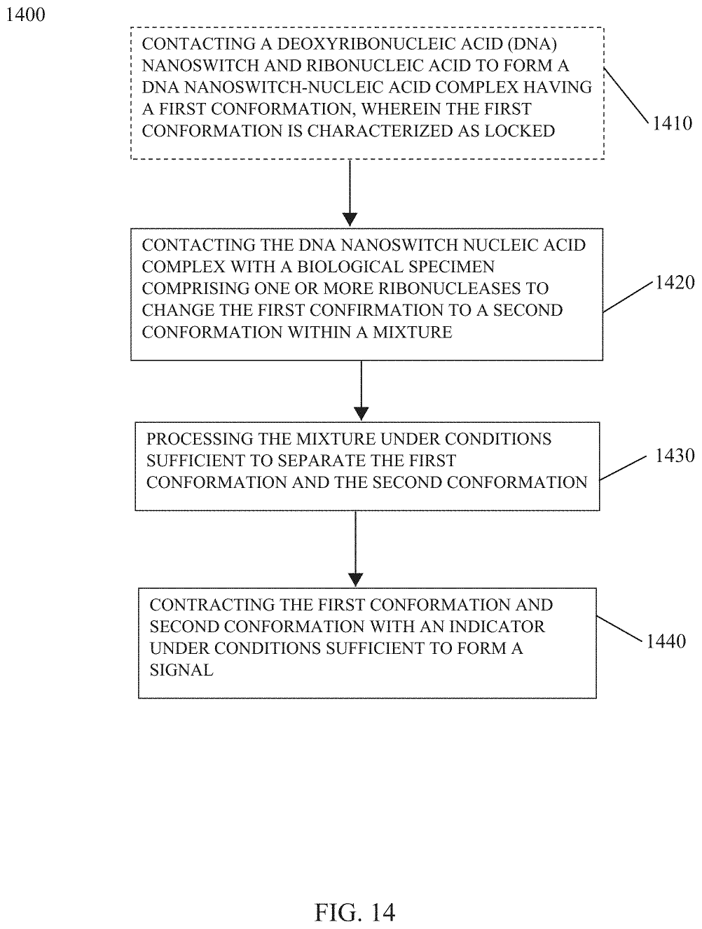

Referring now to FIG. 14, a flowchart of a method 1400 of reconfiguring a nucleic acid construct in accordance with the present disclosure is shown. In embodiments, method 1400 includes at process sequence 1410 contacting a deoxyribonucleic acid (DNA) nanoswitch and ribonucleic acid to form a DNA nanoswitch-nucleic acid complex having a first conformation, wherein the first conformation is characterized as locked or looped. At process sequence 1420, method 1400 includes contacting the DNA nanoswitch-nucleic acid complex with a biological specimen including one or more ribonucleases to change the first conformation to a second conformation within a mixture. At process sequence 1430, method 1400 includes processing a mixture under conditions sufficient to separate the first conformation and the second conformation. At process sequence 1440, method 1400 includes contacting the first conformation and second conformation with an indicator under conditions sufficient to form a signal. In embodiments, the method includes a signal which is predetermined to show a presence or absence of ribonuclease. In embodiments, the signal is predetermined to indicate a code. In embodiments, the method further includes replacing the ribonucleic acid with a deoxyribonucleic acid.

In embodiments, the present disclosure relates to a method of reconfiguring a nucleic acid complex including: contacting a deoxyribonucleic acid (DNA) nanoswitch and a first nucleic acid to form a DNA nanoswitch-nucleic acid complex having a first conformation, wherein the first conformation is characterized as locked; contacting the DNA nanoswitch-nucleic acid complex with a biological specimen to form a mixture, wherein when the first nucleic acid is ribonucleic acid (RNA) and the biological specimen comprises one or more ribonucleases, the first conformation changes to a second conformation characterized as open. In embodiments, the methods further include sequentially processing the mixture under conditions sufficient to separate the first conformation, and when present, the second conformation; and reacting the first conformation, and when present, the second conformation with an indicator under conditions sufficient to form a signal. In embodiments, the first nucleic acid is deoxyribonucleic acid or ribonucleic acid such as an RNA lock of the present disclosure. In embodiments, the first nucleic acid is characterized as an oligonucleotide or polynucleotide having a preselected length. In embodiments, the first nucleic acid binds to the deoxyribonucleic acid (DNA) nanoswitch to form a first conformation comprising a loop.

### EXAMPLE SECTION

### Introduction

Dynamic DNA nanotechnology has yielded a variety of DNA devices and switches that can reconfigure in response to stimuli. (See e.g., Simmel F C, Dittmer W U. DNA Nanodevices. Small. 2005; 1:284-99). Such programmed conformational changes have been used in biosensing (See e.g., Ji W, Li D, Lai W, Yao X, Alam MdF, Zhang W, et al. pH-Operated Triplex DNA Device on MoS2 Nanosheets. Langmuir. 2019; 35:5050-3), mechanical motions (See e.g., Marras A E, Zhou L, Su H-J, Castro C E. Programmable motion of DNA origami mechanisms. Proc Natl Acad Sci. 2015; 112:713-8), and in directing site-specific chemical reactions. (See e.g., Chen Y, Mao C. Reprogramming DNA-Directed Reactions on the Basis of a DNA Conformational Change. J Am Chem Soc. 2004; 126:13240-1).

Through chemical functionalities, DNA nanostructures have been designed to react to triggers such as light (See e.g., Kohman R E, Han X. Light sensitization of DNA nanostructures via incorporation of photo-cleavable spacers. Chem Commun. 2015; 51:5747-50), pH (See e.g., Ji W, Li D, Lai W, Yao X, Alam MdF, Zhang W, et al. pH-Operated Triplex DNA Device on MoS2 Nanosheets. Langmuir. 2019; 35:5050-3), temperature (See e.g., Juul S, lacovelli F, Falconi M, Kragh S L, Christensen B, Frohlich R, et al. Temperature-Controlled Encapsulation and Release of an Active Enzyme in the Cavity of a Self-Assembled DNA Nanocage. ACS Nano. 2013; 7:9724-34) ionic conditions (See e.g., Mao C, Sun W, Shen Z, Seeman N C. A nanomechanical device based on the B-Z transition of DNA. Nature. 1999; 397:144-6) and biological stimuli such as nucleic acids. (See e.g., Chandrasekaran A R, Halvorsen K. Controlled disassembly of a DNA tetrahedron using strand displacement. Nanoscale Adv. 2019; 1:969-72).

DNA nanoswitches that undergo a conformational change in response to an external trigger have been described. These nanoswitches have been used for biosensing of nucleic acids and proteins (See e.g., Chandrasekaran A R, Zavala J, Halvorsen K. Programmable DNA Nanoswitches for Detection of Nucleic Acid Sequences. ACS Sens. 2016; 1:120-3; Chandrasekaran A R, MacIsaac M, Dey P, Levchenko O, Zhou L, Andres M, et al. Cellular microRNA detection with miRacles: microRNA-activated conditional looping of engineered switches. Sci Adv. 2019; 5:eaau9443; Hansen C H, Yang D, Koussa M A, Wong W P. Nanoswitch-linked immunosorbent assay (NLISA) for fast, sensitive, and specific protein detection. Proc Natl Acad Sci. 2017; 114:10367-72; Zhou L, Chandrasekaran A R, Punnoose J A, Bonenfant G, Charles S, Levchenko O, et al. Programmable low-cost DNA-based platform for viral RNA detection. bioRxiv. 2020; 2020.01.12.902452; and Chandrasekaran A R, Dey B K, Halvorsen K. How to Perform miRacles: A Step-by-Step microRNA Detection Protocol Using DNA Nanoswitches. Curr Protoc Mol Biol. 2020; 130:e114), biomolecular interaction analysis (See e.g., Koussa M A, Halvorsen K, Ward A, Wong W P. DNA nanoswitches: a quantitative platform for gel-based biomolecular interaction analysis. Nat Methods. 2015; 12:123-6), single-molecule experimentation (See e.g., Halvorsen K, Schaak D, Wong W P. Nanoengineering a single-molecule mechanical switch using DNA self-assembly. Nanotechnology. 2011; 22:494005; and Yang D, Ward A, Halvorsen K, Wong W P. Multiplexed single-molecule force spectroscopy using a centrifuge. Nat Commun. 2016; 7:11026), and molecular memory (See e.g., Chandrasekaran A R, Levchenko O, Patel D S, MacIsaac M, Halvorsen K. Addressable configurations of DNA nanostructures for rewritable memory. Nucleic Acids Res. 2017; 45:11459-65; Chandrasekaran A R. Reconfigurable DNA Nanoswitches for Graphical Readout of Molecular Signals. ChemBioChem. 2018; 19:1018; and Chandrasekaran A R, Punnoose J A, Valsangkar V, Sheng J, Halvorsen K. Integration of a photocleavable element into DNA nanoswitches. Chem Commun. 2019; 55:6587-90).

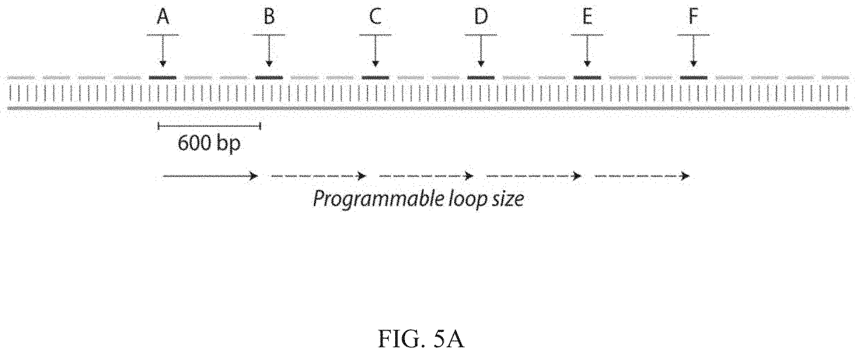

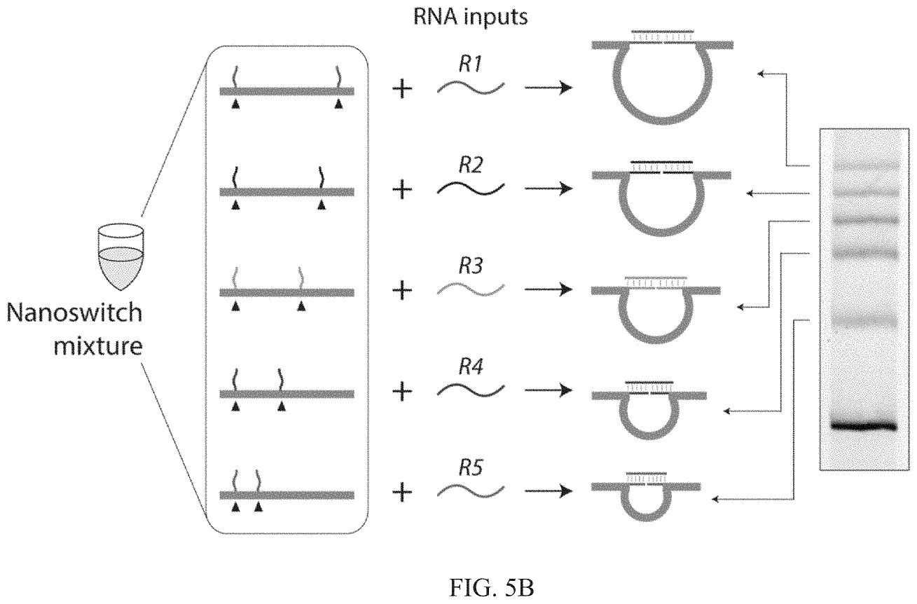

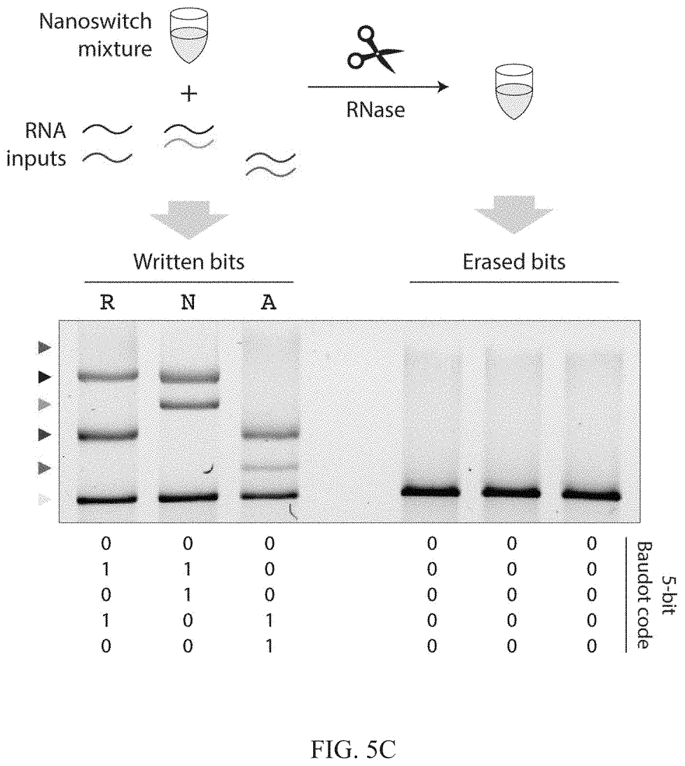

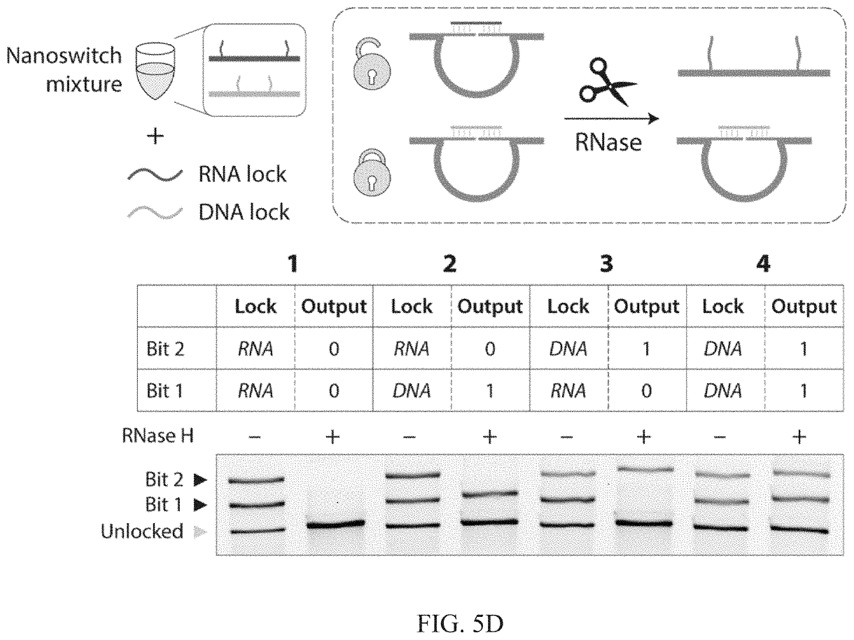

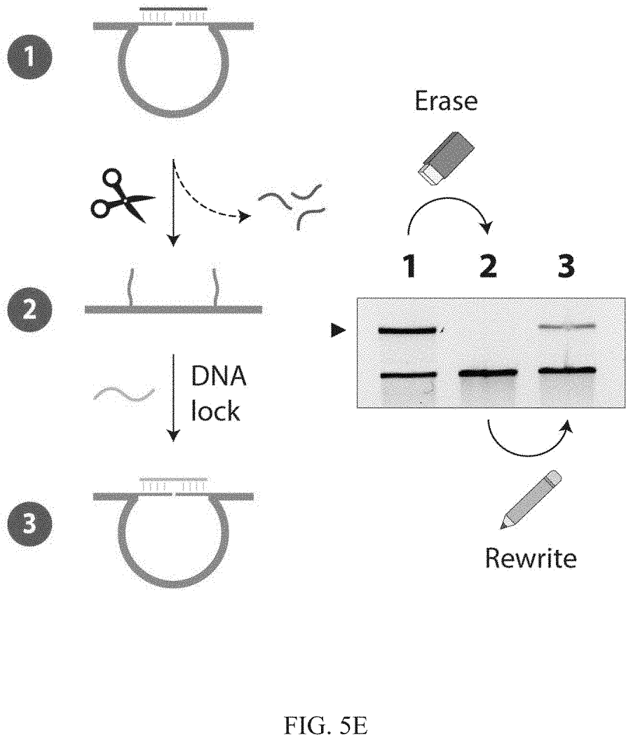

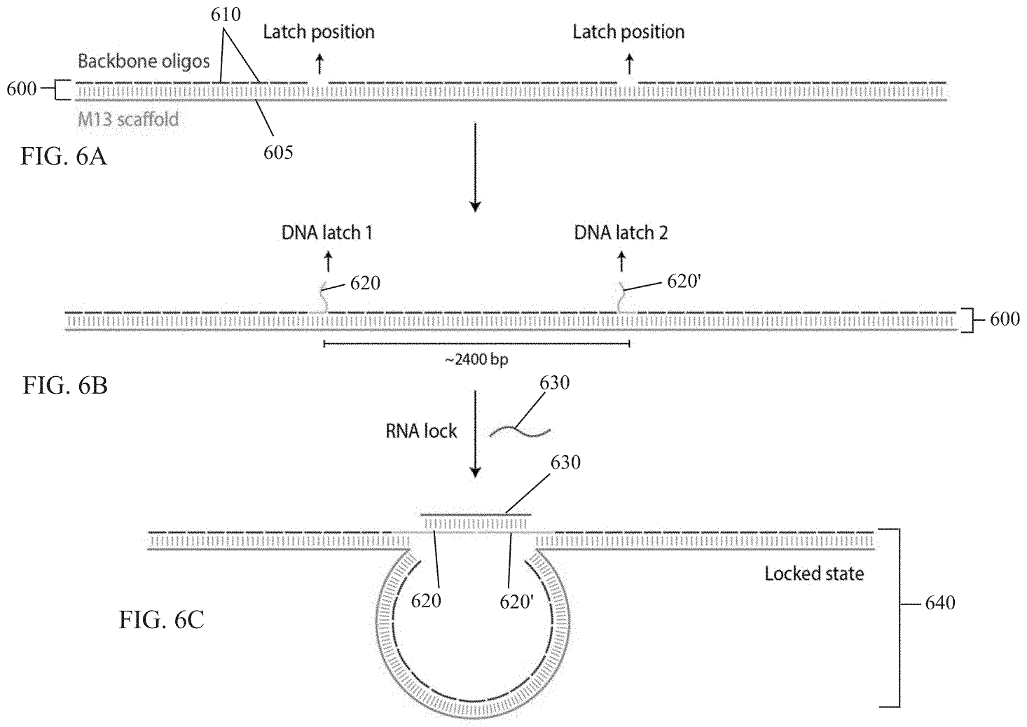

In embodiments, a DNA nanoswitch is a long DNA duplex made from a single-stranded M13 (7249 nucleotides) and short complementary backbone oligos (See e.g., FIG. 2A and FIGS. 6A-6C). In embodiments, specific DNA (See e.g., Chandrasekaran A R, Zavala J, Halvorsen K. Programmable DNA Nanoswitches for Detection of Nucleic Acid Sequences. ACS Sens. 2016; 1:120-3), and RNA (See e.g., Chandrasekaran A R, MacIsaac M, Dey P, Levchenko O, Zhou L, Andres M, et al. Cellular microRNA detection with miRacles: microRNA-activated conditional looping of engineered switches. Sci Adv. 2019; 5:eaau9443) sequences can be targeted by incorporating two complementary single stranded extensions on the nanoswitch, causing target binding to reconfigure the nanoswitch to a looped “locked” state (See e.g., FIG. 6C). The open and locked states of the DNA nanoswitch can be easily visualized on an agarose gel (See e.g., FIGS. 5A-5E). In the example below, DNA nanoswitches are shown that can be triggered by ribonucleases (RNases), and demonstrate two potential applications: (1) detecting RNase activity and (2) in ribonuclease-operated information processing encoded by DNA. The example also includes additional descriptions of the present disclosure.

### Materials and Methods

### Materials

Oligonucleotides were purchased from Integrated DNA Technologies (IDT) with standard desalting. M13 circular DNA, RNase H, RNase T (Exo T), RNase I_{f} and BtsCl enzymes were purchased from New England Biolabs (NEB). GelRed nucleic acid stain was purchased from Biotium, Fremont, Calif., USA. Molecular biology grade agarose was purchased from Fisher BioReagents. The viral genome M13mp18 (7249 nt) was used and preformed constructions of nanoswitches due to commercial availability and frequent use in DNA origami.

### Linearization of M13 DNA

5 μl of 100 nM circular single-stranded M13 DNA, 2.5 μl of 10× Cut Smart buffer, 0.5 μl of 100 μM BtsCl restriction-site complementary-oligonucleotide and 16 μl of deionized water were mixed and annealed from 95° C. to 50° C. in a T100™ Thermal Cycler (Bio-Rad, Hercules, Calif., USA). 1 μl of the BtsCl enzyme (20,000 units/ml) was added to the mixture and incubated at 50° C. for 15 min. The mixture was brought up to 95° C. for 1 min to heat deactivate the enzyme followed by cooling down to 4° C.

### Construction of Nanoswitches

Linearized single-stranded M13 DNA (20 nM) was mixed with ten-fold excess of the backbone oligonucleotides and DNA latches such as the detector oligonucleotides and annealed from 90° C. to 20° C. at 1° C. min⁻¹ in a thermal cycler. Constructed nanoswitches were diluted in 1×PBS to a concentration of 400 pM.

### Construction of DNA-Nanoswitch-Nucleic Acid Construct

To form loops, 2 μl nanoswitches were mixed with the RNA lock strand (typically 2.5 nM final concentration) and incubated at 20° C. overnight.

### RNase H Activity Assay

Locked nanoswitches, such as DNA-nanoswitch-nucleic acid constructs were first mixed with RNase H buffer (1× final) and placed at 37° C. 1 μl of RNase H (different units/μl) was added to 10 μl of locked nanoswitches such as DNA-nanoswitch-nucleic acid constructs and incubated at 37° C. For sensitivity experiments, samples were incubated for 1 hour at 37° C. For time series experiments, RNase H enzyme was added at different time points (from 16 mins to 0 min). After incubation, samples were mixed with 1 μl GelRed (1×final) and 2 μl loading dye (15% Ficoll, 0.1% bromophenol blue), and run on 0.8% agarose gels. A similar protocol was used for the other enzymes.

### Detection in FBS and Cell Lysates

Locked nanoswitches were first mixed with RNase H buffer (1× final), and fetal bovine serum (FBS) or cell lysates were added to a final concentration of 10%. For positive controls, 1 μl of RNase H (5 units/μl) was added to 10 μl of nanoswitch/biofluid mix and incubated at 37° C. for 15 minutes. After incubation, samples were mixed with 1 μl GelRed (1× final) and 2 μl loading dye (15% Ficoll, 0.1% bromophenol blue), and run on 0.8% agarose gels.

### Gel Electrophoresis

Nanoswitches were run in 0.8% agarose gels, cast from molecular biology grade agarose (Fisher BioReagents) dissolved in 0.5×Tris-borate EDTA (TBE). Gels were typically run at 75 V (constant voltage) at room temperature. Samples were pre-stained by mixing 1× GelRed stain with the samples before loading. Gels were imaged with a Bio-Rad Gel Doc XR+gel imager and analyzed using ImageJ.

Nucleic acid sequences suitable for use herein are shown below. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure. Table 1 shows non-limiting examples of suitable backbone oligonucleotides for use in nanoswitch formation of the present disclosure.

TABLE 1
Backbone olegonucleotides

| # | Sequence | Length |
|---|---|---|
| 1 | AGAGCATAAAGCTAAATCGG | 60 |
| | TTGTACCAAAAACATTATGA | |
| | CCCTGTAATACTTTTGCGGG | |
| | (SEQ ID NO: 1) | |
| 2 | AGAAGCCTTTATTTCAACGC | 60 |
| | AAGGATAAAAATTTTTAGAA | |
| | CCCTCATATATTTTAAATGC | |
| | (SEQ ID NO: 2) | |
| 3 | AATGCCTGAGTAATGTGTAG | 60 |
| | GTAAAGATTCAAAAGGGTGA | |
| | GAAAGGCCGGAGACAGTCAA | |
| | (SEQ ID NO: 3) | |
| 4 | ATCACCATCAATATGATATT | 60 |
| | CAACCGTTCTAGCTGATAAA | |
| | TTAATGCCGGAGAGGGTAGC | |
| | (SEQ ID NO: 4) | |
| 5 | TATTTTTGAGAGATCTACAA | 60 |
| | AGGCTATCAGGTCATTGCCT | |
| | GAGAGTCTGGAGCAAACAAG | |
| | (SEQ ID NO: 5) | |
| 6 | AGAATCGATGAACGGTAATC | 60 |
| | GTAAAACTAGCATGTCAATC | |
| | ATATGTACCCCGGTTGATAA | |
| | (SEQ ID NO: 6) | |
| 7 | TCAGAAAAGCCCCAAAAACA | 60 |
| | GGAAGATTGTATAAGCAAAT | |
| | ATTTAAATTGTAAACGTTAA | |
| | (SEQ ID NO: 7) | |
| 8 | TATTTTGTTAAAATTCGCAT | 60 |
| | TAAATTTTTGTTAAATCAGC | |
| | TCATTTTTTAACCAATAGGA | |
| | (SEQ ID NO: 8) | |
| 9 | ACGCCATCAAAAATAATTCG | 60 |
| | CGTCTGGCCTTCCTGTAGCC | |
| | AGCTTTCATCAACATTAAAT | |
| | (SEQ ID NO: 9) | |
| 10 | GGATAGGTCACGTTGGTGTA | 60 |
| | GATGGGCGCATCGTAACCGT | |
| | GCATCTGCCAGTTTGAGGGG | |
| | (SEQ ID NO: 10) | |
| 11 | ACGACGACAGTATCGGCCTC | 60 |
| | AGGAAGATCGCACTCCAGCC | |
| | AGCTTTCCGGCACCGCTTCT | |
| | (SEQ ID NO: 11) | |
| 12 | GGTGCCGGAAACCAGGCAAA | 60 |
| | GCGCCATTCGCCATTCAGGC | |
| | TGCGCAACTGTTGGGAAGGG | |
| | (SEQ ID NO: 12) | |
| 13 | CGATCGGTGCGGGCCTCTTC | 60 |
| | GCTATTACGCCAGCTGGCGA | |
| | AAGGGGGATGTGCTGCAAGG | |
| | (SEQ ID NO: 13) | |
| 14 | CGATTAAGTTGGGTAACGCC | 60 |
| | AGGGTTTTCCCAGTCACGAC | |
| | GTTGTAAAACGACGGCCAGT | |
| | (SEQ ID NO: 14) | |
| 15 | GCCAAGCTTGCATGCCTGCA | 60 |
| | GGTCGACTCTAGAGGATCCC | |
| | CGGGTACCGAGCTCGAATTC | |
| | (SEQ ID NO: 15) | |
| 16 | GTAATCATGGTCATAGCTGT | 60 |
| | TTCCTGTGTGAAATTGTTAT | |
| | CCGCTCACAATTCCACACAA | |
| | (SEQ ID NO: 16) | |
| 17 | CATACGAGCCGGAAGCATAA | 60 |
| | AGTGTAAAGCCTGGGGTGCC | |
| | TAATGAGTGAGCTAACTCAC | |
| | (SEQ ID NO: 17) | |
| 18 | ATTAATTGCGTTGCGCTCAC | 60 |
| | TGCCCGCTTTCCAGTCGGGA | |
| | AACCTGTCGTGCCAGCTGCA | |
| | (SEQ ID NO: 18) | |
| 19 | TTAATGAATCGGCCAACGCG | 60 |
| | CGGGGAGAGGCGGTTTGCGT | |
| | ATTGGGCGCCAGGGTGGTTT | |
| | (SEQ ID NO: 19) | |
| 20 | GTTGCAGCAAGCGGTCCACG | 60 |
| | CTGGTTTGCCCCAGCAGGCG | |
| | AAAATCCTGTTTGATGGTGG | |
| | (SEQ ID NO: 20) | |
| 21 | TTCCGAAATCGGCAAAATCC | 60 |
| | CTTATAAATCAAAAGAATAG | |
| | CCCGAGATAGGGTTGAGTGT | |
| | (SEQ ID NO: 21) | |
| 22 | TGTTCCAGTTTGGAACAAGA | 60 |
| | GTCCACTATTAAAGAACGTG | |
| | GACTCCAACGTCAAAGGGCG | |
| | (SEQ ID NO: 22) | |
| 23 | AAAAACCGTCTATCAGGGCG | 60 |
| | ATGGCCCACTACGTGAACCA | |
| | TCACCCAAATCAAGTTTTTT | |
| | (SEQ ID NO: 23) | |
| 24 | GGGGTCGAGGTGCCGTAAAG | 60 |
| | CACTAAATCGGAACCCTAAA | |
| | GGGAGCCCCCGATTTAGAGC | |
| | (SEQ ID NO: 24) | |
| 25 | TTGACGGGGAAAGCCGGCGA | 60 |
| | ACGTGGCGAGAAAGGAAGGG | |
| | AAGAAAGCGAAAGGAGCGGG | |
| | (SEQ ID NO: 25) | |
| 26 | CGCTAGGGCGCTGGCAAGTG | 60 |
| | TAGCGGTCACGCTGCGCGTA | |
| | ACCACCACACCCGCCGCGCT | |
| | (SEQ ID NO: 26) | |
| 27 | TAATGCGCCGCTACAGGGCG | 60 |
| | CGTACTATGGTTGCTTTGAC | |
| | GAGCACGTATAACGTGCTTT | |
| | (SEQ ID NO: 27) | |
| 28 | CCTCGTTAGAATCAGAGCGG | 60 |
| | GAGCTAAACAGGAGGCCGAT | |
| | TAAAGGGATTTTAGACAGGA | |
| | (SEQ ID NO: 28) | |
| 29 | ACGGTACGCCAGAATCCTGA | 60 |
| | GAAGTGTTTTTATAATCAGT | |
| | GAGGCCACCGAGTAAAAGAG | |
| | (SEQ ID NO: 29) | |
| 30 | TTGCCTGAGTAGAAGAACTC | 60 |
| | AAACTATCGGCCTTGCTGGT | |
| | AATATCCAGAACAATATTAC | |
| | (SEQ ID NO: 30) | |
| 31 | CGCCAGCCATTGCAACAGGA | 60 |
| | AAAACGCTCATGGAAATACC | |
| | TACATTTTGACGCTCAATCG | |
| | (SEQ ID NO: 31) | |
| 32 | TCTGAAATGGATTATTTACA | 60 |
| | TTGGCAGATTCACCAGTCAC | |
| | ACGACCAGTAATAAAAGGGA | |
| | (SEQ ID NO: 32) | |
| 33 | CATTCTGGCCAACAGAGATA | 60 |
| | GAACCCTTCTGACCTGAAAG | |
| | CGTAAGAATACGTGGCACAG | |
| | (SEQ ID NO: 33) | |
| 34 | ACAATATTTTTGAATGGCTA | 60 |
| | TTAGTCTTTAATGCGCGAAC | |
| | TGATAGCCCTAAAACATCGC | |
| | (SEQ ID NO: 34) | |
| 35 | CATTAAAAATACCGAACGAA | 60 |
| | CCACCAGCAGAAGATAAAAC | |
| | AGAGGTGAGGCGGTCAGTAT | |
| | (SEQ ID NO: 35) | |
| 36 | TAACACCGCCTGCAACAGTG | 60 |
| | CCACGCTGAGAGCCAGCAGC | |
| | AAATGAAAAATCTAAAGCAT | |
| | (SEQ ID NO: 36) | |
| 37 | CACCTTGCTGAACCTCAAAT | 60 |
| | ATCAAACCCTCAATCAATAT | |
| | CTGGTCAGTTGGCAAATCAA | |
| | (SEQ ID NO: 37) | |
| 38 | CAGTTGAAAGGAATTGAGGA | 60 |
| | AGGTTATCTAAAATATCTTT | |
| | AGGAGCACTAACAACTAATA | |
| | (SEQ ID NO: 38) | |
| 39 | GATTAGAGCCGTCAATAGAT | 60 |
| | AATACATTTGAGGATTTAGA | |
| | AGTATTAGACTTTACAAACA | |
| | (SEQ ID NO: 39) | |
| 40 | CATTATCATTTTGCGGAACA | 60 |
| | AAGAAACCACCAGAAGGAGC | |
| | GGAATTATCATCATATTCCT | |
| | (SEQ ID NO: 40) | |
| 41 | GATTATCAGATGATGGCAAT | 60 |
| | TCATCAATATAATCCTGATT | |
| | GTTTGGATTATACTTCTGAA | |
| | (SEQ ID NO: 41) | |
| 42 | TAATGGAAGGGTTAGAACCT | 60 |
| | ACCATATCAAAATTATTTGC | |
| | ACGTAAAACAGAAATAAAGA | |
| | (SEQ ID NO: 42) | |
| 43 | AATTGCGTAGATTTTCAGGT | 60 |
| | TTAACGTCAGATGAATATAC | |
| | AGTAACAGTACCTTTTACAT | |
| | (SEQ ID NO: 43) | |
| 44 | CGGGAGAAACAATAACGGAT | 60 |
| | TCGCCTGATTGCTTTGAATA | |
| | CCAAGTTACAAAATCGCGCA | |
| | (SEQ ID NO: 44) | |
| 45 | GAGGCGAATTATTCATTTCA | 60 |
| | ATTACCTGAGCAAAAGAAGA | |
| | TGATGAAACAAACATCAAGA | |
| | (SEQ ID NO: 45) | |
| 46 | AAACAAAATTAATTACATTT | 60 |
| | AACAATTTCATTTGAATTAC | |
| | CTTTTTTAATGGAAACAGTA | |
| | (SEQ ID NO: 46) | |
| 47 | CATAAATCAATATATGTGAG | 60 |
| | TGAATAACCTTGCTTCTGTA | |
| | AATCGTCGCTATTAATTAAT | |
| | (SEQ ID NO: 47) | |
| 48 | TTTCCCTTAGAATCCTTGAA | 60 |
| | AACATAGCGATAGCTTAGAT | |
| | TAAGACGCTGAGAAGAGTCA | |
| | (SEQ ID NO: 48) | |
| 49 | ATAGTGAATTTATCAAAATC | 60 |
| | ATAGGTCTGAGAGACTACCT | |
| | TTTTAACCTCCGGCTTAGGT | |
| | (SEQ ID NO: 49) | |
| 50 | GAAAACTTTTTCAAATATAT | 60 |
| | TTTAGTTAATTTCATCTTCT | |
| | GACCTAAATTTAATGGTTTG | |
| | (SEQ ID NO: 50) | |
| 51 | AAATACCGACCGTGTGATAA | 60 |
| | ATAAGGCGTTAAATAAGAAT | |
| | AAACACCGGAATCATAATTA | |
| | (SEQ ID NO: 51) | |
| 52 | CTAGAAAAAGCCTGTTTAGT | 60 |
| | ATCATATGCGTTATACAAAT | |
| | TCTTACCAGTATAAAGCCAA | |
| | (SEQ ID NO: 52) | |
| 53 | CGCTCAACAGTAGGGCTTAA | 60 |
| | TTGAGAATCGCCATATTTAA | |
| | CAACGCCAACATGTAATTTA | |
| | (SEQ ID NO: 53) | |
| 54 | GGCAGAGGCATTTTCGAGCC | 60 |
| | AGTAATAAGAGAATATAAAG | |
| | TACCGACAAAAGGTAAAGTA | |
| | (SEQ ID NO: 54) | |
| 55 | ATTCTGTCCAGACGACGACA | 60 |
| | ATAAACAACATGTTCAGCTA | |
| | ATGCAGAACGCGCCTGTTTA | |
| | (SEQ ID NO: 55) | |
| 56 | TCAACAATAGATAAGTCCTG | 60 |
| | AACAAGAAAAATAATATCCC | |
| | ATCCTAATTTACGAGCATGT | |
| | (SEQ ID NO: 56) | |
| 57 | AGAAACCAATCAATAATCGG | 60 |
| | CTGTCTTTCCTTATCATTCC | |
| | AAGAACGGGTATTAAACCAA | |
| | (SEQ ID NO: 57) | |
| 58 | GTACCGCACTCATCGAGAAC | 60 |
| | AAGCAAGCCGTTTTTATTTT | |
| | CATCGTAGGAATCATTACCG | |
| | (SEQ ID NO: 58) | |
| 59 | CGCCCAATAGCAAGCAAATC | 60 |
| | AGATATAGAAGGCTTATCCG | |
| | GTATTCTAAGAACGCGAGGC | |
| | (SEQ ID NO: 59) | |
| 60 | ATTTTGCACCCAGCTACAAT | 60 |
| | TTTATCCTGAATCTTACCAA | |
| | CGCTAACGAGCGTCTTTCCA | |
| | (SEQ ID NO: 60) | |
| 61 | GAGCCTAATTTGCCAGTTAC | 60 |
| | AAAATAAACAGCCATATTAT | |
| | TTATCCCAATCCAAATAAGA | |
| | (SEQ ID NO: 61) | |
| 62 | AACGATTTTTTGTTTAACGT | 60 |
| | CAAAAATGAAAATAGCAGCC | |
| | TTTACAGAGAGAATAACATA | |
| | (SEQ ID NO: 62) | |
| 63 | AAAACAGGGAAGCGCATTAG | 60 |
| | ACGGGAGAATTAACTGAACA | |
| | CCCTGAACAAAGTCAGAGGG | |
| | (SEQ ID NO: 63) | |
| 64 | TAATTGAGCGCTAATATCAG | 60 |
| | AGAGATAACCCACAAGAATT | |
| | GAGTTAAGCCCAATAATAAG | |
| | (SEQ ID NO: 64) | |
| 65 | AGCAAGAAACAATGAAATAG | 60 |
| | CAATAGCTATCTTACCGAAG | |
| | CCCTTTTTAAGAAAAGTAAG | |
| | (SEQ ID NO: 65) | |
| 66 | CAGATAGCCGAACAAAGTTA | 60 |
| | CCAGAAGGAAACCGAGGAAA | |
| | CGCAATAATAACGGAATACC | |
| | (SEQ ID NO: 66) | |
| 67 | CAAAAGAACTGGCATGATTA | 60 |
| | AGACTCCTTATTACGCAGTA | |
| | TGTTAGCAAACGTAGAAAAT | |
| | (SEQ ID NO: 67) | |
| 68 | ACATACATAAAGGTGGCAAC | 60 |
| | ATATAAAAGAAACGCAAAGA | |
| | CACCACGGAATAAGTTTATT | |
| | (SEQ ID NO: 68) | |
| 69 | TTGTCACAATCAATAGAAAA | 60 |
| | TTCATATGGTTTACCAGCGC | |
| | CAAAGACAAAAGGGCGACAT | |
| | (SEQ ID NO: 69) | |
| 70 | TCACCGTCACCGACTTGAGC | 60 |
| | CATTTGGGAATTAGAGCCAG | |
| | CAAAATCACCAGTAGCACCA | |
| | (SEQ ID NO: 70) | |
| 71 | TTACCATTAGCAAGGCCGGA | 60 |
| | AACGTCACCAATGAAACCAT | |
| | CGATAGCAGCACCGTAATCA | |
| | (SEQ ID NO: 71) | |
| 72 | GTAGCGACAGAATCAAGTTT | 60 |
| | GCCTTTAGCGTCAGACTGTA | |
| | GCGCGTTTTCATCGGCATTT | |
| | (SEQ ID NO: 72) | |
| 73 | TCGGTCATAGCCCCCTTATT | 60 |
| | AGCGTTTGCCATCTTTTCAT | |
| | AATCAAAATCACCGGAACCA | |
| | (SEQ ID NO: 73) | |
| 74 | GAGCCACCACCGGAACCGCC | 60 |
| | TCCCTCAGAGCCGCCACCCT | |
| | CAGAACCGCCACCCTCAGAG | |
| | (SEQ ID NO: 74) | |
| 75 | CCACCACCCTCAGAGCCGCC | 60 |
| | ACCAGAACCACCACCAGAGC | |
| | CGCCGCCAGCATTGACAGGA | |
| | (SEQ ID NO: 75) | |
| 76 | GGTTGAGGCAGGTCAGACGA | 60 |
| | TTGGCCTTGATATTCACAAA | |
| | CAAATAAATCCTCATTAAAG | |
| | (SEQ ID NO: 76) | |
| 77 | CCAGAATGGAAAGCGCAGTC | 60 |
| | TCTGAATTTACCGTTCCAGT | |
| | AAGCGTCATACATGGCTTTT | |
| | (SEQ ID NO: 77) | |
| 78 | GATGATACAGGAGTGTACTG | 60 |
| | GTAATAAGTTTTAACGGGGT | |
| | CAGTGCCTTGAGTAACAGTG | |
| | (SEQ ID NO: 78) | |
| 79 | CCCGTATAAACAGTTAATGC | 60 |
| | CCCCTGCCTATTTCGGAACC | |
| | TATTATTCTGAAACATGAAA | |
| | (SEQ ID NO: 79) | |
| 80 | CCAGGCGGATAAGTGCCGTC | 60 |
| | GAGAGGGTTGATATAAGTAT | |
| | AGCCCGGAATAGGTGTATCA | |
| | (SEQ ID NO: 80) | |
| 81 | CCGTACTCAGGAGGTTTAGT | 60 |
| | ACCGCCACCCTCAGAACCGC | |
| | CACCCTCAGAACCGCCACCC | |
| | (SEQ ID NO: 81) | |
| 82 | TCAGAGCCACCACCCTCATT | 60 |
| | TTCAGGGATAGCAAGCCCAA | |
| | TAGGAACCCATGTACCGTAA | |
| | (SEQ ID NO: 82) | |
| 83 | CACTGAGTTTCGTCACCAGT | 60 |
| | ACAAACTACAACGCCTGTAG | |
| | CATTCCACAGACAGCCCTCA | |
| | (SEQ ID NO: 83) | |
| 84 | TAGTTAGCGTAACGATCTAA | 60 |
| | AGTTTTGTCGTCTTTCCAGA | |
| | CGTTAGTAAATGAATTTTCT | |
| | (SEQ ID NO: 84) | |
| 85 | GTATGGGATTTTGCTAAACA | 60 |
| | ACTTTCAACAGTTTCAGCGG | |
| | AGTGAGAATAGAAAGGAACA | |
| | (SEQ ID NO: 85) | |
| 86 | ACTAAAGGAATTGCGAATAA | 60 |
| | TAATTTTTTCACGTTGAAAA | |
| | TCTCCAAAAAAAAGGCTCCA | |
| | (SEQ ID NO: 86) | |
| 87 | AAAGGAGCCTTTAATTGTAT | 60 |
| | CGGTTTATCAGCTTGCTTTC | |
| | GAGGTGAATTTCTTAAACAG | |
| | (SEQ ID NO: 87) | |
| 88 | CTTGATACCGATAGTTGCGC | 60 |
| | CGACAATGACAACAACCATC | |
| | GCCCACGCATAACCGATATA | |
| | (SEQ ID NO: 88) | |
| 89 | TTCGGTCGCTGAGGCTTGCA | 60 |
| | GGGAGTTAAAGGCCGCTTTT | |
| | GCGGGATCGTCACCCTCAGC | |
| | (SEQ ID NO: 89) | |
| 90 | CTTTTTCATGAGGAAGTTTC | 60 |
| | CATTAAACGGGTAAAATACG | |
| | TAATGCCACTACGAAGGCAC | |
| | (SEQ ID NO: 90) | |
| 91 | CAACCTAAAACGAAAGAGGC | 60 |
| | AAAAGAATACACTAAAACAC | |
| | TCATCTTTGACCCCCAGCGA | |
| | (SEQ ID NO: 91) | |
| 92 | TTATACCAAGCGCGAAACAA | 60 |
| | AGTACAACGGAGATTTGTAT | |
| | CATCGCCTGATAAATTGTGT | |
| | (SEQ ID NO: 92) | |
| 93 | CGAAATCCGCGACCTGCTCC | 60 |
| | ATGTTACTTAGCCGGAACGA | |
| | GGCGCAGACGGTCAATCATA | |
| | (SEQ ID NO: 93) | |
| 94 | AGGGAACCGAACTGACCAAC | 60 |
| | TTTGAAAGAGGACAGATGAA | |
| | CGGTGTACAGACCAGGCGCA | |
| | (SEQ ID NO: 94) | |
| 95 | TAGGCTGGCTGACCTTCATC | 60 |
| | AAGAGTAATCTTGACAAGAA | |
| | CCGGATATTCATTACCCAAA | |
| | (SEQ ID NO: 95) | |
| 96 | TCAACGTAACAAAGCTGCTC | 60 |
| | ATTCAGTGAATAAGGCTTGC | |
| | CCTGACGAGAAACACCAGAA | |
| | (SEQ ID NO: 96) | |
| 97 | CGAGTAGTAAATTGGGCTTG | 60 |
| | AGATGGTTTAATTTCAACTT | |
| | TAATCATTGTGAATTACCTT | |
| | (SEQ ID NO: 97) | |
| 98 | ATGCGATTTTAAGAACTGGC | 60 |
| | TCATTATACCAGTCAGGACG | |
| | TTGGGAAGAAAAATCTACGT | |
| | (SEQ ID NO: 98) | |
| 99 | TAATAAAACGAACTAACGGA | 60 |
| | ACAACATTATTACAGGTAGA | |
| | AAGATTCATCAGTTGAGATT | |
| | (SEQ ID NO: 99) | |
| 100 | TAAGAGCAACACTATCATAA | 60 |
| | CCCTCGTTTACCAGACGACG | |
| | ATAAAAACCAAAATAGCGAG | |
| | (SEQ ID NO: 100) | |
| 101 | AGGCTTTTGCAAAAGAAGTT | 60 |
| | TTGCCAGAGGGGGTAATAGT | |
| | AAAATGTTTAGACTGGATAG | |
| | (SEQ ID NO: 101) | |
| 102 | CGTCCAATACTGCGGAATCG | 60 |
| | TCATAAATATTCATTGAATC | |
| | CCCCTCAAATGCTTTAAACA | |
| | (SEQ ID NO: 102) | |
| 103 | GTTCAGAAAACGAGAATGAC | 60 |
| | CATAAATCAAAAATCAGGTC | |
| | TTTACCCTGACTATTATAGT | |
| | (SEQ ID NO: 103) | |
| 104 | CAGAAGCAAAGCGGATTGCA | 60 |
| | TCAAAAAGATTAAGAGGAAG | |
| | CCCGAAAGACTTCAAATATC | |
| | (SEQ ID NO: 104) | |
| 105 | GCGTTTTAATTCGAGCTTCA | 60 |
| | AAGCGAACCAGACCGGAAGC | |
| | AAACTCCAACAGGTCAGGAT | |
| | (SEQ ID NO: 105) | |
| 106 | TAGAGAGTACCTTTAATTGC | 60 |
| | TCCTTTTGATAAGAGGTCAT | |
| | TTTTGCGGATGGCTTAGAGC | |
| | {SEQIDNO:106) | |
| 107 | TTAATTGOTGAATATAATGC | 60 |
| | TGTAGCTCAACATGTTTTAA | |
| | ATATGCAACTAAAGTACGGT | |
| | (SEQ ID NO: 107) | |
| 108 | GTCTGGAAGTTTCATTCCAT | 60 |
| | ATAACAGTTGATTCCCAATT | |
| | CTGCGAACGAGTAGATTTAG | |
| | (SEQ ID NO: 108) | |
| 109 | TTTGACCATTAGATACATTT | 49 |
| | CGCAAATGGTCAATAACCTG | |
| | TTTAGCTATfSEQIDNO:109) | |
| 110 | AACATCCAATAAATCATACA | 60 |
| | GGCAAGGCAAAGAATTAGCA | |
| | AAATTAAGCAATAAAGCCTC | |
| | (SEQ ID NO: 110) | |
| 111 | GTGAGCGAGTAACAACCCGT | 60 |
| | CGGATTCTCCGTGGGAACAA | |
| | ACGGCGGATTGACCGTAATG | |
| | (SEQ ID NO: 111) | |
| 112 | TTCTTTTCACCAGTGAGACG | 60 |
| | GGCAACAGCTGATTGCCCTT | |
| | CACCGCCTGGCCCTGAGAGA | |
| | (SEQ ID NO: 112) | |
| 113 | AGCGAAAGACAGCATCGGAA | 60 |
| | CGAGGGTAGCAACGGCTACA | |
| | GAGGCTTTGAGGACTAAAGA | |
| | (SEQ ID NO: 113) | |
| 114 | TAGGAATACCACATTCAACT | 60 |
| | AATGCAGATACATAACGCCA | |
| | AAAGGAATTACGAGGCATAG | |
| | (SEQ ID NO: 114) | |
| 115 | ATTTTCATTTGGGGCGCGAG | 60 |
| | CTGAAAAGGTGGCATCAATT | |
| | CTACTAATAGTAGTAGCATT | |
| | (SEQ ID NO: 115) | |

Table 2 shows non-limiting examples of suitable latch position holders for use in nanoswitch formation of the present disclosure. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure.

TABLE 2

| # | Sequence | Length |
|---|---|---|
| Holder A | TCTGTCCATCACGCAAATT | 60 |
| | AACCGTTGTAGCAATACTT | |
| | CTTTGATTAGTAATAACAT | |
| | CAC | |
| | (SEQ ID NO: 116) | |
| Holder B | ATTCGACAACTCGTATTAA | 60 |
| | ATCCTTTGCCCGAACGTTA | |
| | TTAATTTTAAAAGTTTGAG | |
| | TAA | |
| | (SEQ ID NO: 117) | |
| Holder C | TGGGTTATATAACTATATG | 60 |
| | TAAATGOTGATGCAAATCC | |
| | AATCGCAAGACAAAGAACG | |
| | CGA | |
| | (SEQ ID NO: 118) | |
| Holder D | GTTTTAGCGAACCTCCCGA | 60 |
| | CTTGCGGGAGGTTTTGAAG | |
| | CCTTAAATCAAGATTAGT | |
| | TGCT | |
| | (SEQ ID NO: 119) | |
| Holder E | TCAACCGATTGAGGGAGGG | 60 |
| | AAGGTAAATATTGACGGAA | |
| | ATTATTCATTAAAGGTGAA | |
| | TTA | |
| | (SEQ ID NO: 120) | |
| Holder F | GTATTAAGAGGCTGAGACT | 60 |
| | CCTCAAGAGAAGGATTAGG | |
| | ATTAGCGGGGTTTTGCTCA | |
| | GTA | |
| | (SEQ ID NO: 121) | |

Table 3 shows non-limiting examples of filler sequences for use in nanoswitch formation of the present disclosure. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure.

TABLE 3
Filler sequences

| # | Sequence | Length |
|---|---|---|
| FA | TCTGTCCATC | 20 |
| | ACGCAAATTA | |
| | (SEQ ID | |
| | NO: 122) | |
| FB | AATTTTAAAA | 20 |
| | GTTTGAGTAA | |
| | (SEQ ID | |
| | NO: 123) | |
| FC | TCGCAAGACA | 20 |
| | AAGAACGCGA | |
| | (SEQ ID | |
| | NO: 124) | |
| FD | TTAAATCAAG | 20 |
| | ATTAGTTGCT | |
| | (SEQ ID | |
| | NO: 125) | |
| FE | TATTCATTAA | 20 |
| | AGGTGAATTA | |
| | (SEQ ID | |
| | NO: 126) | |
| FF | TAGCGGGGTT | 20 |
| | TTGCTCAGTA | |
| | (SEQ ID | |
| | NO: 127) | |

Table 4 shows non-limiting examples of DNA latches for use in nanoswitch formation of the present disclosure. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure. Segments or regions in bold indicate locations where detector strands are placed. Single underlined and double underlined segments or regions are single stranded extensions (latches) that are complementary to two halves of the lock strands (also coded by single underlining and double underlining). Latches shown in bold were used for all experiments where a single nanoswitch was used.

TABLE 4
DNA latches

| # | Sequence | Length |
|---|---|---|
| Latch A- | ACCGTTGTAGCAATACTTCT | 51 |
| R1 | TTGATTAGTAATAACATCAC | |
| | CAAGCTGATTT | |
| | (SEQ ID NO: 128) | |
| Latch B- | ACACCCGGTGAATTCGACAA | 51 |
| R1 | CTCGTATTAAATCCTTTGCC | |
| | CGAACGTTATT | |
| | (SEQ ID NO: 129) | |
| Latch A- | ACCGTTGTAGCAATACTTCT | 51 |
| R2 | TTGATTAGTAATAACATCAC | |
| | CGCCAATATTT | |
| | (SEQ ID NO: 130) | |
| Latch C- | ACGTGCTGCTATGGGTTATA | 51 |
| R2 | TAACTATATGTAAATGCTGA | |
| | TGCAAATCCAA | |
| | (SEQ ID NO: 131) | |
| Latch A- | ACCGTTGTAGCAATACTTCT | 50 |
| R3 | TTGATTAGTAATAACATCAC | |
| | CCAACAACAT | |
| | (SEQ ID NO: 132) | |
| Latch D- | GAAACTACCTAGTTTTAGCG | 51 |
| R3 | AACCTCCCGACTTGCGGGAG | |
| | GTTTTGAAGCC | |
| | (SEQ ID NO: 133) | |
| Latch A- | ACCGTTGTAGCAATACTTCT | 51 |
| R4 | TTGATTAGTAATAACATCAC | |
| | AACCACACAAC | |
| | (SEQ ID NO: 134) | |
| Latch E- | CTACTACCTCATCAACCGAT | 51 |
| R4 | TGAGGGAGGGAAGGTAAATA | |
| | TTGACGGAAAT | |
| | (SEQ ID NO: 135) | |
| Latch A- | ACCGTTGTAGCAATACTTCT | 51 |
| R5 | TTGATTAGTAATAACATCAC | |
| | CCACACACTTC | |
| | (SEQ ID NO: 136) | |
| Latch F- | CTTACATTCCAGTATTAAGA | 51 |
| R5 | GGCTGAGACTCCTCAAGAGA | |
| | AGGATTAGGAT | |
| | (SEQ ID NO: 137) | |

Table 5 shows non-limiting examples of RNA locks for use in nanoswitch formation of the present disclosure. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure.

TABLE 5
RNA locks

| # | Sequence | Length |
|---|---|---|
| R1 | UCACCGGGUGUAAAUCAGCUUG | 22 |
| | (SEQ ID NO: 138) | |
| R2 | UAGCAGCACGUAAAUAUUGGCG | 22 |
| | (SEQ ID NO: 139) | |
| R3 | UAGGUAGUUUCAUGUUGUUGG | 21 |
| | (SEQ ID NO: 140) | |
| R4 | UGAGGUAGUAGGUUGUGUGGUU | 22 |
| | (SEQ ID NO: 141) | |
| R5 | UGGAAUGUAAGGAAGUGUGUGG | 22 |
| | (SEQ ID NO: 142) | |

Table 6 shows non-limiting examples of strands or segments for use in nanoswitch formation of the present disclosure. It is noted that all sequences are written from 5′ to 3′. In embodiments, it is contemplated that nucleic acid sequences have at least 90%, at least 95%, at least 97%, or at least 99% sequence identity to the sequences identified herein may be suitable for use in accordance with the present disclosure.

TABLE 6
Other strands

| # | Sequence | Length |
|---|---|---|
| D4 | TGAGGTAGTAGGTTGTGTGGTT | 22 |
| (rewrite) | (SEQ ID NO: 143) | |
| BtsCI cut | CTACTAATAGTAGTAGCATTAA | 40 |
| site | CATCCAATAAATCATA | |
| oligo | CA(SEQ ID NO: 144) | |

### Ribonuclease-Responsive DNA Nanoswitches

A DNA nanoswitch that can be reconfigured or undergo conformational change using ribonucleases (RNases) and use in two applications: biosensing and molecular computing was explored. For biosensing, detection of RNase H and other RNases was shown in relevant biological fluids and temperatures, as well as inhibition by the known enzyme inhibitor kanamycin. For molecular computing, RNases can be used to enable erasing, write protection, and erase-rewrite functionality for information-encoding DNA nanoswitches. The simplistic mix-and-read nature of the ribonuclease-activated DNA nanoswitches could facilitate use in assays for identifying RNase contamination in biological samples or for screening and characterization of RNase inhibitors.

In embodiments, a DNA nanoswitch is a long DNA duplex made from a single-stranded M13 (7249 nucleotides) and short complementary backbone oligos (see for example FIG. 3A and FIGS. 6A-6C).

Referring to FIG. 3A-3C, DNA nanoswitch design and operation in accordance with embodiments of the present disclosure is shown. Referring to FIG. 3A, a nanoswitch 320′ is locked in a looped conformation with a pre-hybridized RNA lock strand, nucleic acid, or nucleic acid-of-interest 310. A latch strand as described above is shown binding to a scaffold segment. In embodiments, the RNA lock strand, nucleic acid, or nucleic acid-of-interest has a predetermined length, or includes a predetermined number of nucleotide units. In embodiments, the RNA lock strand, nucleic acid, or nucleic acid-of-interest is characterized as an oligonucleotide. On the addition of RNase H 330, the lock strand or nucleic acid-of-interest 310 is digested, resulting in unlooping of the nanoswitch 320 to the open state. Referring to FIG. 3B. the mechanism of cleavage of an RNA lock strand or nucleic acid-of-interest 310 by RNase H and release of the DNA latches or detector strand 325 and 325′ is shown. Referring to FIG. 3C, a DNA nanoswitch 320 is locked by an RNA lock strand 321 into a looped conformation 326. RNase H 330 causes cleavage of the RNA lock or nucleic acid-of-interest 310, causing the nanoswitch to unlock and reconfigure into the linear state as shown at (3). In embodiments, the conformational change from a looped conformation to a linear state can be readout on an agarose gel 345 to detect the presence of RNase H (inset).

In embodiments, the present disclosure provides a signal-off strategy based on RNase-triggered reconfiguration of DNA nanoswitches. To accomplish this, a DNA nanoswitch is prehybridized with an RNA lock strand, forming a locked nanoswitch (FIG. 3A, locked looped state). For proof-of-concept, RNase H, a ribonuclease that specifically catalyzes the hydrolysis of RNA in the RNA/DNA duplex was tested. On the addition of RNase H, the RNA lock is digested, leading to release of the DNA latches and thus opening of the nanoswitch (FIG. 3B). This DNA nanoswitch conformational change provides a direct gel-based readout of the RNase H activity (FIG. 3C). The signal is provided by the intercalation of thousands of dye molecules (from regular DNA gel stains) into the nanoswitch, thus providing a huge signal even for a single molecular event such as enzymatic cleavage of an RNA strand (FIG. 3C, inset). The use of a long scaffold DNA such as the M13 provides higher number of intercalation sites for these DNA stains and provides enhanced signal compared to shorter scaffolds.

Referring to FIG. 6A the nanoswitch 600 is a duplex formed from linear M13 605 and short complementary backbone oligonucleotides 610. As shown in FIG. 6B, two DNA latches or detector strands 620 and 620′ containing single-stranded overhangs that complement an RNA lock are placed at two locations on the scaffold. As shown in FIG. 6C, an RNA lock or nucleic acid-of-interest 630 is shown bound to the detector strands 620 and 620′ forming a DNA nanoswitch-nucleic acid complex 640 of the present disclosure.

### Results and Discussion

The detection of RNase activity is demonstrated herein. RNases are involved in many biological processes including neurotoxicity, genome replication and maintenance, angiogenic activity, immune-suppressivity and antitumor activity. (See e.g., Sorrentino S. Human extracellular ribonucleases: multiplicity, molecular diversity and catalytic properties of the major RNase types. Cell Mol Life Sci. 1998; 54:785-94). In retroviruses such as HIV1, an RNase H activity associated with the viral reverse transcriptase is required for replication, making RNase H inhibitors potential drugs for AIDS (See e.g., Boyer P L, Smith S J, Zhao X Z, Das K, Gruber K, Arnold E, et al. Developing and Evaluating Inhibitors against the RNase H Active Site of HIV-1 Reverse Transcriptase. J Virol [Internet]. 2018 [cited 2020 Mar. 24]; 92. Available from: https://jvi.asm.org/content/92/13/e02203-17). RNases are also potential biomarkers for neoplastic diseases such as pancreatic cancer and in cystic fibrosis. (See e.g., Huang W, Zhao M, Wei N, Wang X, Cao H, Du Q, et al. Site-Specific RNase A Activity Was Dramatically Reduced in Serum from Multiple Types of Cancer Patients. PLOS ONE. 2014; 9:e96490). In a laboratory setting, RNases are important for some molecular biology protocols, but can also be the source of frustrating contaminations that degrade biological RNA samples. Detection of RNases and their inhibition have therefore become increasingly important, and various RNase detection kits are commercially available. Early methods developed to determine RNase activity include renaturation gel assays (See e.g., Frank P, Cazenave C, Albert S, Toulme J J. Sensitive Detection of Low Levels of Ribonuclease H Activity by an Improved Renaturation Gel Assay. Biochem Biophys Res Commun. 1993; 196:1552-7), high-performance liquid chromatography (HPLC) (See e.g., Hogrefe H H, Hogrefe R I, Walder R Y, Walder J A. Kinetic analysis of Escherichia coli RNase H using DNA-RNA-DNA/DNA substrates. J Biol Chem. 1990; 265:5561-6), colorimetry (See e.g., Xie X, Xu W, Li T, Liu X. Colorimetric Detection of HIV-1 Ribonuclease H Activity by Gold Nanoparticles. Small. 2011; 7:1393-6), and fluorometry (See e.g., Lee C Y, Jang H, Park K S, Park H G. A label-free and enzyme-free signal amplification strategy for a sensitive RNase H activity assay. Nanoscale. 2017; 9:16149-53). These methods suffer from limitations such as complexity, high-cost and low sensitivity (See e.g., Sato S, Takenaka S. Highly Sensitive Nuclease Assays Based on Chemically Modified DNA or RNA. Sensors. 2014; 14:12437-50), spurring recent detection approaches using catalytic hairpin assembly (See e.g., Lee C Y, Jang H, Park K S, Park H G. A label-free and enzyme-free signal amplification strategy for a sensitive RNase H activity assay. Nanoscale. 2017; 9:16149-53), gold nanoparticle conjugates (See e,g, m Kim J H, Estabrook R A, Braun G, Lee B R, Reich N O. Specific and sensitive detection of nucleic acids and RNases using gold nanoparticle-RNA-fluorescent dye conjugates. Chem Commun. 2007; 4342-4), magnetic nanoparticles (See e,g., Persano S, Vecchio G, Pompa P P. A hybrid chimeric system for versatile and ultra-sensitive RNase detection. Sci Rep. 2015; 5:1-5) and DNA walkers (See e.g. Wang Y, Hu N, Liu C, Nie C, He M, Zhang J, et al. An RNase H-powered DNA walking machine for sensitive detection of RNase H and the screening of related inhibitors. Nanoscale. 2020; 12:1673-9). These assays have higher sensitivity but problematically include multiple wash steps, additional amplification, indirect quantitation and specific equipment for readout.

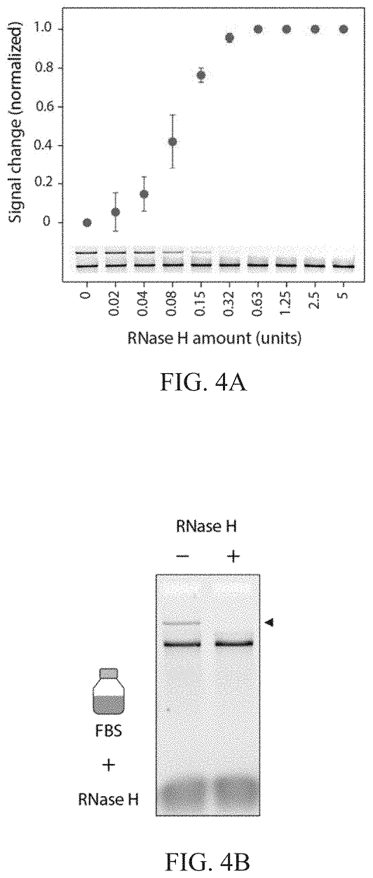

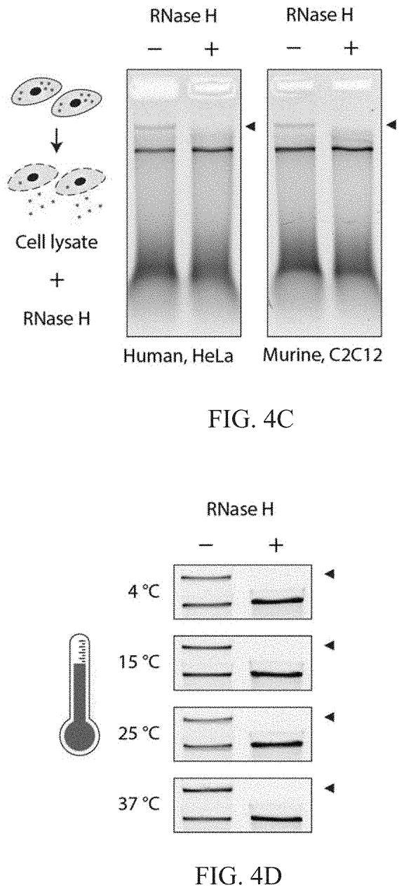

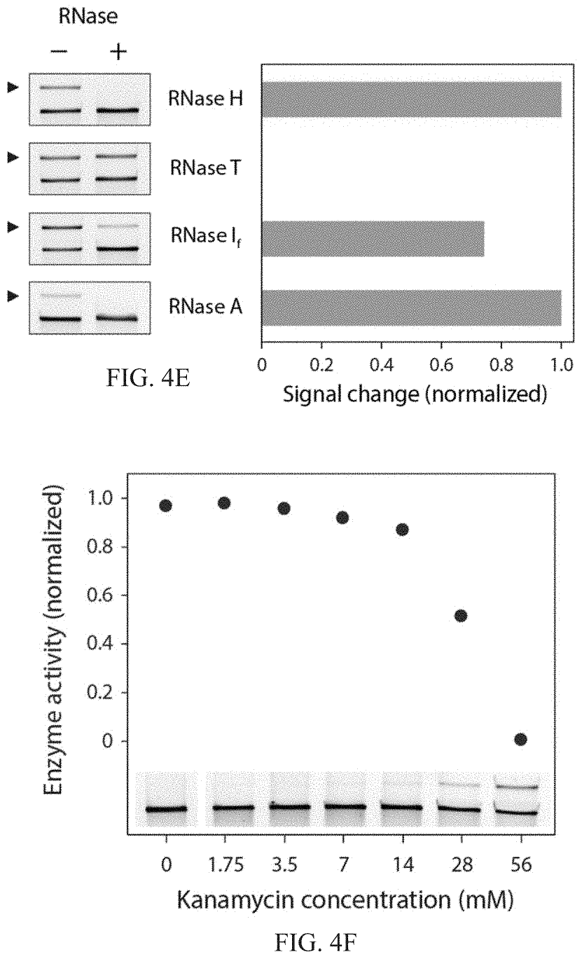

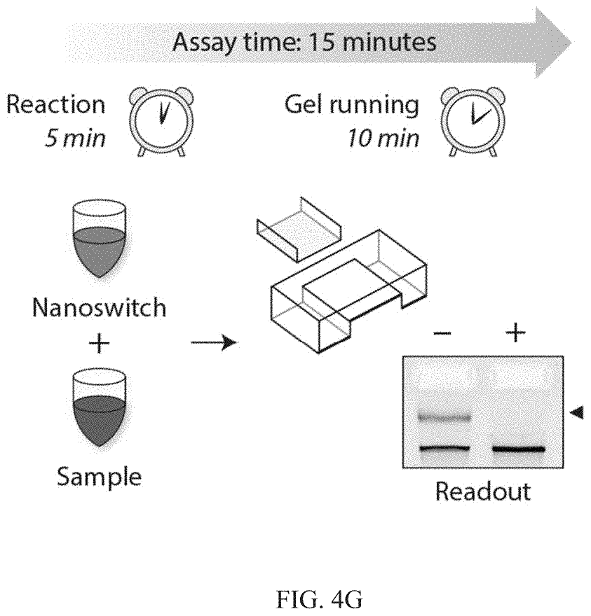

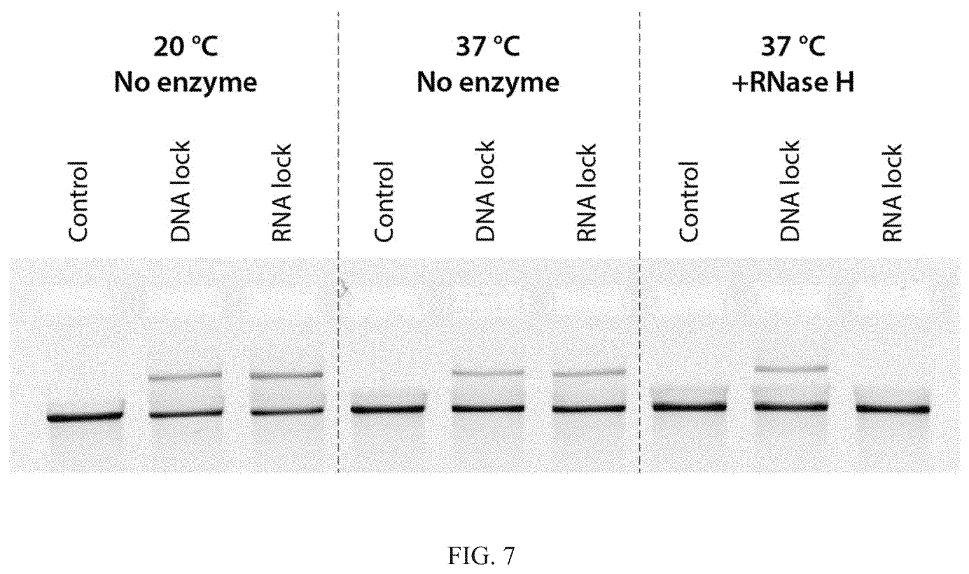

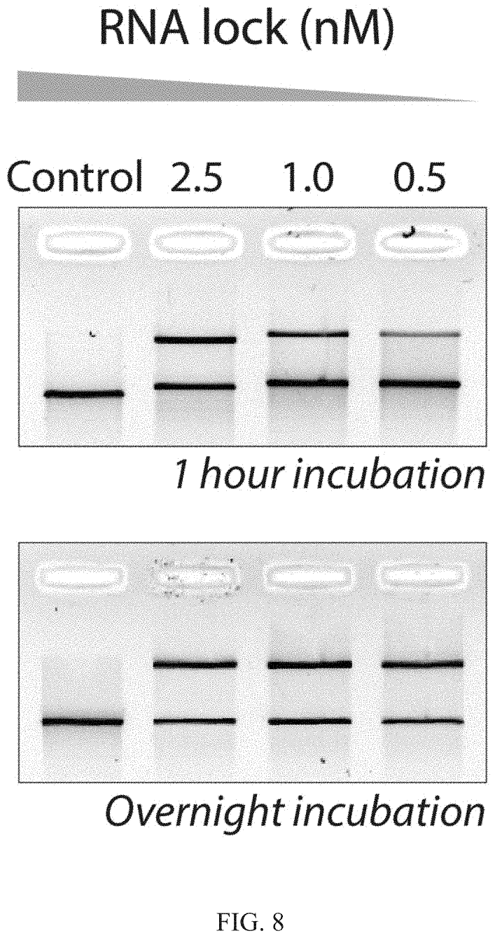

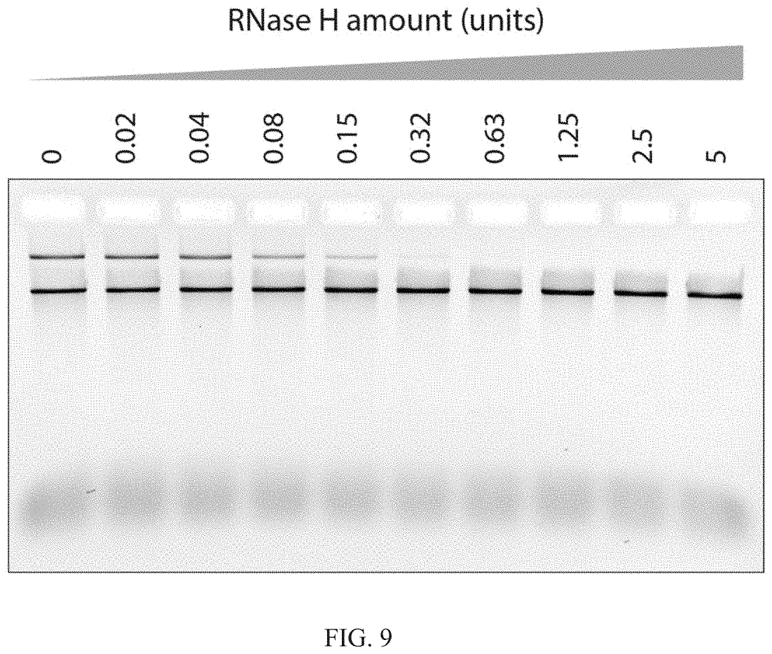

Along with demonstrating the basic operation of the DNA nanoswitches by RNase H in FIGS. 3A, 3B and 3C, controls were performed to show that looped nanoswitches were not affected at the optimal RNase H temperature of 37° C. and that nanoswitches locked with DNA strands (nucleic acids-of-interest including DNA oligonucleotides) were not affected by RNase H (FIG. 7). FIG. 7 shows various electrophoresis readouts showing RNase H activity with an RNA lock (RNA nucleic acid-of-interest) on the DNA nanoswitch but is inactive when a DNA lock (DNA nucleic acid-of-interest) is used. Further, looping efficiency of the nanoswitches was tested with different concentrations of the RNA lock (RNA nucleic acids-of-interest) at different incubation times, with average looping yields of 60% (FIG. 8). FIG. 8 shows various electrophoresis readouts for optimizing RNA locking of DNA nanoswitches with different concentrations of RNA lock strand and varying incubation times. Further, the sensitivity of the assay was tested with different amounts of RNase H in a 1-hour assay and showed corresponding variation in unlooping of the nanoswitch (FIG. 4A and FIG. 9). For example, FIG. 9 is an electrophoresis readout showing unlooping of DNA nanoswitch-nucleic acid complexes with different amounts of RNase H enzyme. By quantifying the reduction in the looped band of the nanoswitch we reliably detected as low as 0.02 units of RNase H in a 10 μl reaction.

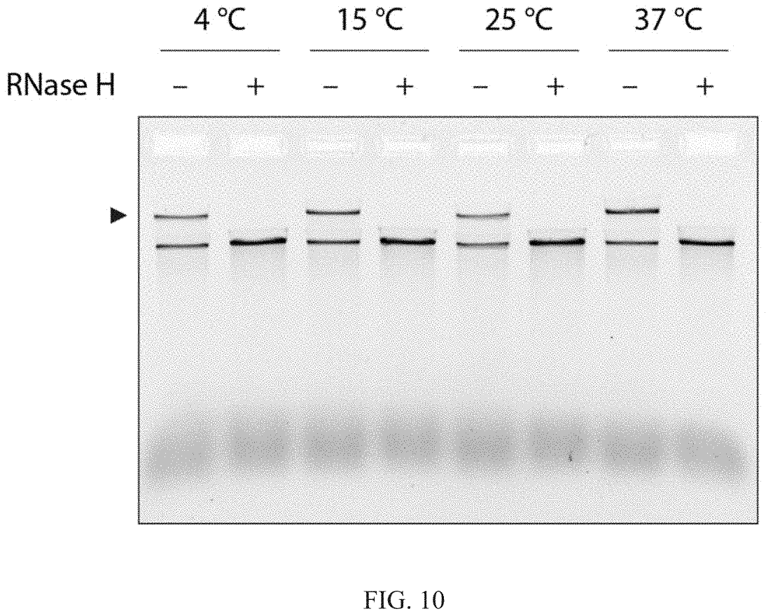

As a step toward testing ribonuclease presence in biological samples, a known concentration of RNase H was spiked into fetal bovine serum (FBS) and cell lysates extracted from human (HeLa) and murine (C2C12) cell lines. RNase detection was preserved in 10% FBS and cell lysates with the unlocking of the DNA nanoswitch in both biofluids (FIGS. 4B-4C). The assay was demonstrated under different temperatures ranging from 4° C. to 37° C., suggesting that the assay could be performed without any temperature control (FIG. 4D and FIG. 10). To further generalize the assay, a panel of other RNases was tested: RNase T, RNase I_{f} and RNase A. The response of the locked nanoswitches vary between these RNases, with RNase H and RNase A showing high levels of unlooping, RNase I_{f} with partial unlooping, and RNase T showing no activity on the nanoswitch (see e.g., FIG. 4E).

Next, the potential of assays of the present disclosure was tested for screening RNase H inhibitors. Since HIV1 reverse transcriptase is known to have an RNase activity, HIV drug development includes the screening of small molecules and antibiotics that can inhibit this enzymatic activity. As a proof-of-concept demonstration, a known RNase H inhibitor kanamycin was tested for its inhibitory effects on RNase H. (See e.g., Zhao C, Fan J, Peng L, Zhao L, Tong C, Wang W, et al. An end-point method based on graphene oxide for RNase H analysis and inhibitors screening. Biosens Bioelectron. 2017; 90:103-9). The nanoswitch was incubated with different concentrations of kanamycin and then added RNase H (0.5 U) (FIG. 4F). Quantified results show that kanamycin inhibits the RNA cleavage activity of the enzyme, with an IC50 value of 30.6 mM. The inhibition efficiency of kanamycin reported in literature has varied in levels from weak to strong inhibition of RNase H (See e.g. Zhao C, Fan J, Peng L, Zhao L, Tong C, Wang W, et al. An end-point method based on graphene oxide for RNase H analysis and inhibitors screening. Biosens Bioelectron. 2017; 90:103-9; and Hu N, Wang Y, Liu C, He M, Nie C, Zhang J, et al. An enzyme-initiated DNAzyme motor for RNase H activity imaging in living cell. Chem Commun. 2020; 56:639-42), and the inhibition level reported here is within the extremes of the reported numbers. To further make a rapid readout, a start-to-finish assay was performed within 15 mins, by incubating the nanoswitches with RNase H for 5 mins followed by agarose gel electrophoresis for 10 mins (FIG. 4G). The assay of the present disclosure can also be used in point-of-care setting with existing buffer-less electrophoresis units (eg: E-Gel from Thermo Fisher).

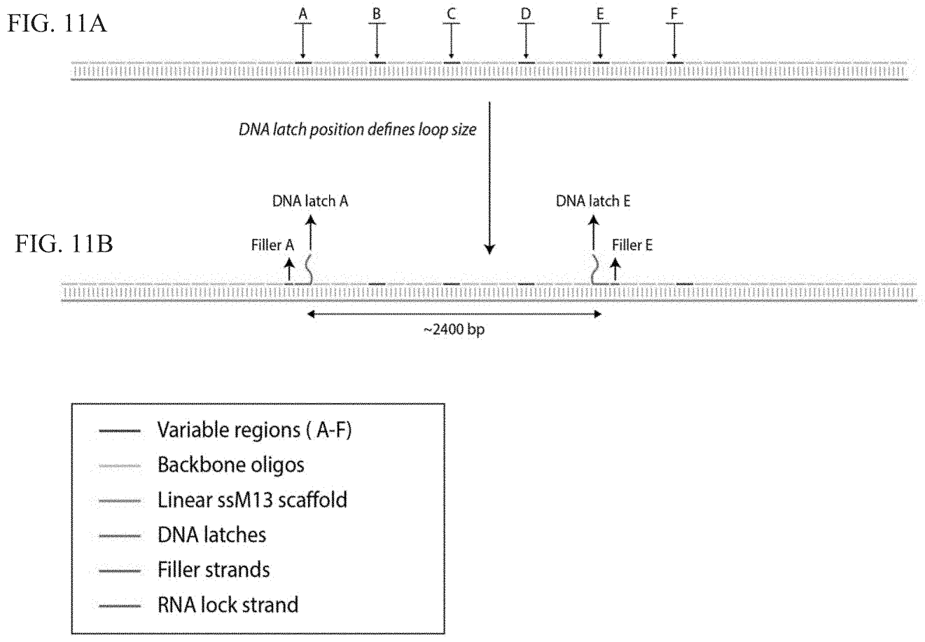

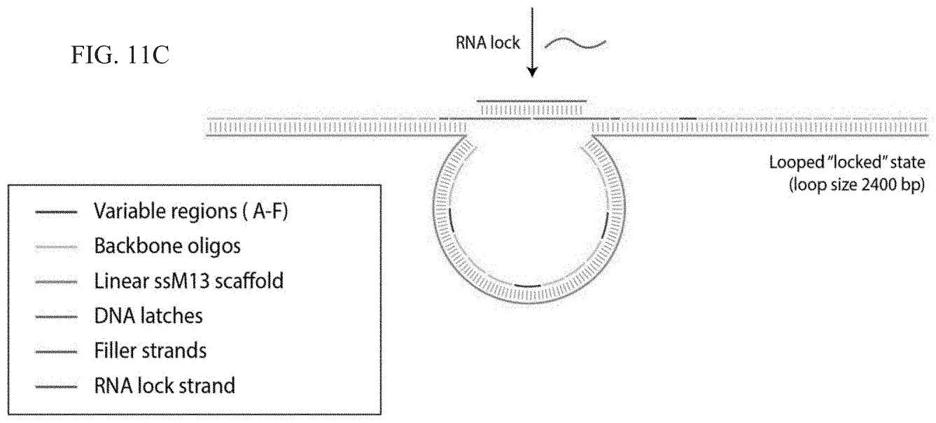

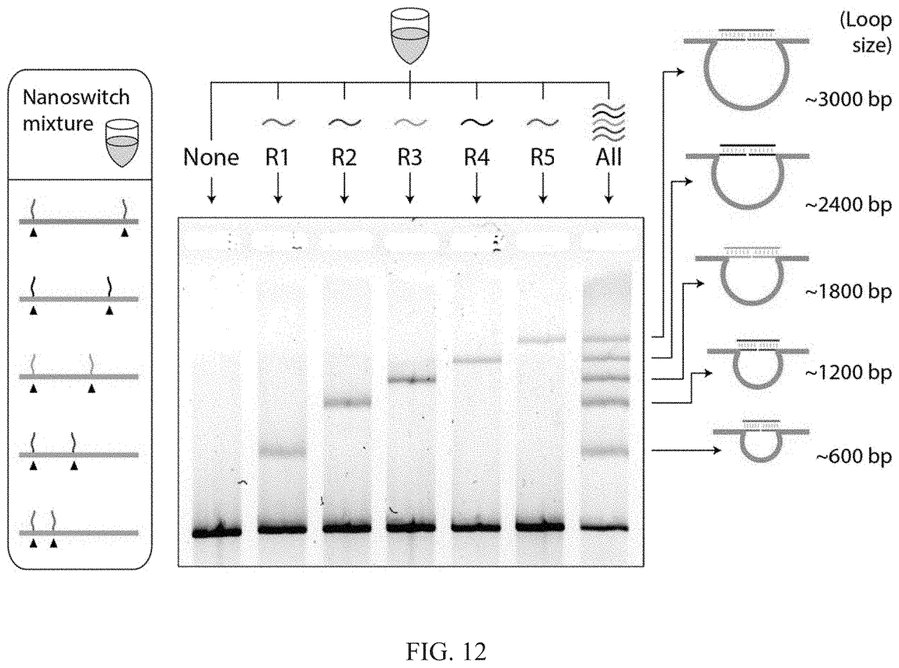

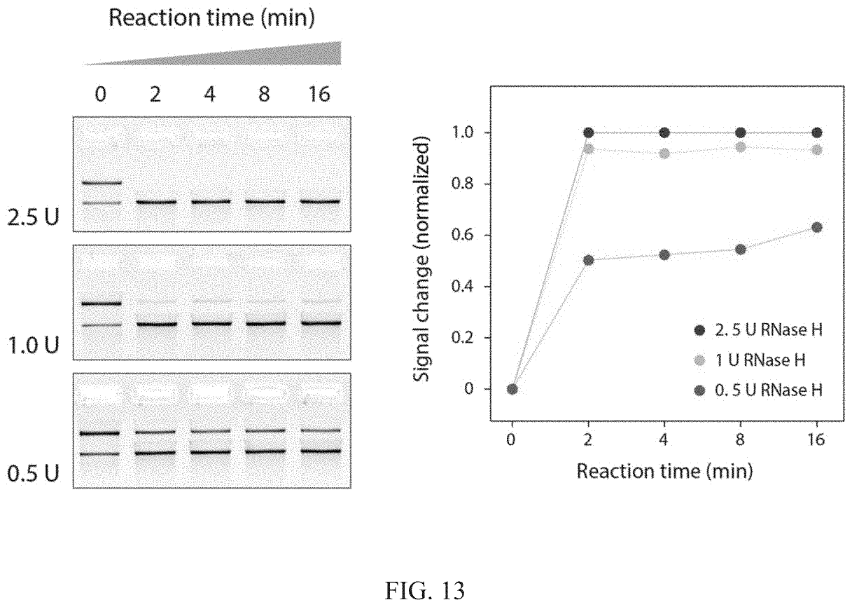

For another application, the use of RNase H was demonstrated in processing information encoded using DNA nanoswitches. Nanoswitches of different loop sizes may be configured to encode bits of memory. (See e.g., Chandrasekaran A R, Levchenko O, Patel D S, MacIsaac M, Halvorsen K. Addressable configurations of DNA nanostructures for rewritable memory. Nucleic Acids Res. 2017; 45:11459-65; and Chandrasekaran A R. Reconfigurable DNA Nanoswitches for Graphical Readout of Molecular Signals. ChemBioChem. 2018; 19:1018-21) (both of which are entirely incorporated herein by reference). Embodiments, of the present disclosure expand the control of erasable and rewritable memory using a ribonuclease. By incorporating DNA latches such as detector oligonucleotides formed of nucleic acids-of-interest at defined locations on the scaffold, the resulting loop size of the locked conformation can be changed (FIG. 5A and FIG. 11). In embodiments, the present disclosure provides a nanoswitch mix containing nanoswitches that can form different loop sizes such as up to five different loop sizes. In embodiments, specific lock strands (nucleic acids-of-interest) bind sequence-specifically to corresponding nanoswitches in the mixture and cause reconfiguration to form loops of different sizes based on the distance between the DNA latches (FIG. 5B). The location of the DNA latches along the scaffold in each nanoswitch was designed or preselected to provide well-separated locked bands on the gel when all five RNA locks are present (FIG. 12). The kinetics of the writing process can be tuned by changing the loop size of the DNA nanoswitches, with shorter loops forming faster than longer loops. (See e.g., Chandrasekaran A R, Levchenko O, Patel D S, MacIsaac M, Halvorsen K. Addressable configurations of DNA nanostructures for rewritable memory. Nucleic Acids Res. 2017; 45:11459-65).

Reacting the nanoswitches with RNA inputs (20-23 nucleotides) to get the highest looping for each of the 5 nanoswitches in the mix (typically overnight) is shown at FIG. 8. The length regime for target nucleic acid results in better looping of nanoswitches. (See e.g., Chandrasekaran A R, Zavala J, Halvorsen K. Programmable DNA Nanoswitches for Detection of Nucleic Acid Sequences. ACS Sens. 2016; 1:120-3).

Using the 5 different locked states of the nanoswitches, a memory system was created that can encode 5 bits of information per gel lane, which can be translated into alphabet characters using the 5-bit Baudot code. (See e.g., Detlefsen G D, Kerr R H. Baudot code. In: Encyclopedia of Computer Science. GBR: John Wiley and Sons Ltd.; 2003. p. 134-13).

Specific RNA lock strands or preselected nucleic acids-of-interest were used as inputs to trigger specific nanoswitches, with each loop acting as a bit in a 5-bit code (shortest loop is bit 1 and longest loop is bit 5, FIG. 12). On a gel-based readout, each lane was treated as a 5-bit encoder, with multiple consecutive gel lanes providing a string of characters. To demonstrate this, the characters “RNA” were displayed on a gel by encoded information using RNA lock strands of the present disclosure (FIG. 5C). RNase H was used to act as a molecular eraser, cleaving the RNA in a DNA/RNA hybrid to erase the written information.

Next, it was showed that using specific RNA or DNA lock strands, could “protect” certain bits from being erased. A mix containing two nanoswitches with different loop sizes was used, and used combinations of DNA or RNA locks to demonstrate this write protection feature. Once the inputs are added, there were two written bits corresponding to the bands for two loop sizes (FIG. 5D). On adding RNase H, only the RNA locked strand was cleaved, changing that nanoswitch to the open state (FIG. 5D, inset). Even within the same mixture, nanoswitches locked by a DNA strand were not affected by the RNase H. All four possible combinations of the DNA and RNA lock strands were demonstrated and showed that the bits written using a DNA input strand is “protected” against erasing.

In addition to writing and erasing capabilities, molecular memory systems also often require a rewriting functionality. Once the RNA locks are cleaved by the enzyme, the DNA latches on the nanoswitches are again available for hybridization. To demonstrate rewriting, the bits using RNase H were erased and one rewrote the bits using DNA locks of the same sequence (FIG. 5E). For erasing using RNase H, a time series was performed showing that erasing pre-written bits can be completed in under a few mins with >1 unit/μl of RNase H (FIG. 13). This processing time is faster than erasing encoded bits using toehold-based DNA strand displacement (See e.g., Chandrasekaran A R, Levchenko O, Patel D S, MacIsaac M, Halvorsen K. Addressable configurations of DNA nanostructures for rewritable memory. Nucleic Acids Res. 2017; 45:11459-65) or light-activation of photocleavable locks. (See e.g., Chandrasekaran A R, Punnoose J A, Valsangkar V, Sheng J, Halvorsen K. Integration of a photocleavable element into DNA nanoswitches. Chem Commun. 2019; 55:6587-90). These experiments show that one can controllably encode information using DNA nanoswitches, erase information using an enzyme such as RNase H, and further rewrite information as required.

The DNA nanoswitch and complexes described herein are a versatile biomolecular platform with broad applications. (See e.g., Chandrasekaran A R, MacIsaac M, Dey P, Levchenko O, Zhou L, Andres M, et al. Cellular microRNA detection with miRacles: microRNA-activated conditional looping of engineered switches. Sci Adv. 2019; 5:eaau9443; Zhou L, Chandrasekaran A R, Punnoose J A, Bonenfant G, Charles S, Levchenko O, et al. Programmable low-cost DNA-based platform for viral RNA detection. bioRxiv. 2020; 2020.01.12.902452; Chandrasekaran A R, Dey B K, Halvorsen K. How to Perform miRacles: A Step-by-Step microRNA Detection Protocol Using DNA Nanoswitches. Curr Protoc Mol Biol. 2020; 130:e114; Koussa M A, Halvorsen K, Ward A, Wong W P. DNA nanoswitches: a quantitative platform for gel-based biomolecular interaction analysis. Nat Methods. 2015; 12:123-6; and Chandrasekaran A R, Levchenko O, Patel D S, MacIsaac M, Halvorsen K. Addressable configurations of DNA nanostructures for rewritable memory. Nucleic Acids Res. 2017; 45:11459-65).

The use of RNase provides an additional tool to manipulate DNA nanoswitches. DNA nanoswitch assay adds to the suite of available techniques for monitoring RNase activity. Most of these assays require fluorescently-labeled probes and depend on a separate signal amplification step to enhance the signal produced by RNA cleavage. (See e.g., Lee C Y, Jang H, Park K S, Park H G. A label-free and enzyme-free signal amplification strategy for a sensitive RNase H activity assay. Nanoscale. 2017; 9:16149-53; and Kim J H, Estabrook R A, Braun G, Lee B R, Reich N O. Specific and sensitive detection of nucleic acids and RNases using gold nanoparticle-RNA-fluorescent dye conjugates. Chem Commun. 2007; 4342-4). In contrast, a ˜7 kbp long nanoswitch provides an inherent signal by the intercalation of thousands of dye molecules from regular DNA stains, providing a high signal even for the cleavage of a single RNA lock by RNases (unlooping the nanoswitch causes the shift of thousands of dye molecules on a gel). For use in a biological context, biostability of DNA nanoswitches is an important factor. Detection of nucleic acid and protein biomarkers in 10-20% serum is possible (See e.g., Chandrasekaran A R, Zavala J, Halvorsen K. Programmable DNA Nanoswitches for Detection of Nucleic Acid Sequences. ACS Sens. 2016; 1:120-3; and Hansen C H, Yang D, Koussa M A, Wong W P. Nanoswitch-linked immunosorbent assay (NLISA) for fast, sensitive, and specific protein detection. Proc Natl Acad Sci. 2017; 114:10367-72), as well as in human urine (See e.g., Zhou L, Chandrasekaran A R, Punnoose J A, Bonenfant G, Charles S, Levchenko O, et al. Programmable low-cost DNA-based platform for viral RNA detection. bioRxiv. 2020; 2020.01.12.902452), showing the inherent stability of DNA nanoswitches for use in real-life applications. Results in FBS and cell lysates demonstrated in this work further establish the potential of the DNA nanoswitch assay in in vitro applications. In molecular computing, this enzyme-based operation of DNA nanoswitches adds to our suite of nanoswitches that are responsive to light or DNA (See e.g., Chandrasekaran A R, Levchenko O, Patel D S, MacIsaac M, Halvorsen K. Addressable configurations of DNA nanostructures for rewritable memory. Nucleic Acids Res. 2017; 45:11459-65; and Chandrasekaran A R, Punnoose J A, Valsangkar V, Sheng J, Halvorsen K. Integration of a photocleavable element into DNA nanoswitches. Chem Commun. 2019; 55:6587-90) opening up avenues to create DNA devices that can be orthogonally operated by physical (light), biological (RNA/DNA/enzymes), or chemical (pH) triggers. For RNase detection, an assay of the present disclosure provides a simple and effective mix-and-read strategy with a gel-based read-out. The strategy does not require labeling and amplification, thus being easy to adapt by any lab without the need for expensive equipment. It is believed it fills an important need for identifying RNase contamination in biological samples, and for characterizing RNase inhibitors.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed:

1. A method of reconfiguring a nucleic acid complex comprising:
contacting a deoxyribonucleic acid (DNA) nanoswitch and a first nucleic acid to form a DNA nanoswitch-nucleic acid complex having a first conformation, wherein the first conformation is characterized as locked;
contacting the DNA nanoswitch-nucleic acid complex with a biological specimen to form a mixture, wherein when the first nucleic acid is ribonucleic acid (RNA) and the biological specimen comprises one or more ribonucleases, the first conformation changes to a second conformation characterized as open;
processing the mixture under conditions sufficient to separate the first conformation, and when present, the second conformation; and
reacting the first conformation, and when present, the second conformation with an indicator under conditions sufficient to form a signal.

2. The method of claim 1, wherein the first nucleic acid is deoxyribonucleic acid or ribonucleic acid.

3. The method of claim 1, wherein the first nucleic acid is characterized as an oligonucleotide or polynucleotide having a preselected length.

4. The method of claim 1, wherein the first nucleic acid binds to the deoxyribonucleic acid (DNA) nanoswitch to form a first conformation comprising a loop.

5. The method of claim 4, wherein the loop has a preselected size.

6. The method of claim 1, wherein the biological specimen comprises one or more ribonucleases.

7. The method of claim 6, wherein the one or more ribonucleases comprises an endonuclease capable of producing one or more 5′ phophomonoesters.

8. The method of claim 7, wherein the endonuclease is ribonuclease H.

9. The method of claim 1, wherein processing the mixture comprises electrophoresing the mixture under conditions sufficient to separate the first conformation and the second conformation.

10. The method of claim 1, wherein the first nucleic acid is deoxyribonucleic acid (DNA), and wherein the first nucleic acid binds to the deoxyribonucleic acid (DNA) nanoswitch to form a first conformation comprising a loop, wherein the loop is characterized as unchanging in a presence of one or more ribonucleases.

11. The method of claim 1, wherein a formation of the second conformation signals a presence of one or more ribonucleases (RNases).

12. The method of claim 1, wherein the second conformation has an altered functionality compared to the first conformation.

13. The method of claim 1, wherein the first nucleic acid is an oligonucleotide configured to hold the deoxyribonucleic acid (DNA) nanoswitch in the first conformation, and wherein the oligonucleotide is configured to provide a code.

14. The method of claim 1, wherein the first nucleic acid is a DNA oligonucleotide configured to hold the deoxyribonucleic acid (DNA) nanoswitch in the first conformation, and wherein the DNA oligonucleotide is configured to maintain the first conformation in a presence of ribonuclease.

15. The method of claim 1, wherein the first conformation is configured to change to a second conformation when contacted with target-of-interest, and wherein the second conformation is configured to report a target-of-interest.

16. A method of reconfiguring a polynucleotide comprising:
contacting a deoxyribonucleic acid (DNA) nanoswitch and a first ribonucleic acid to form a DNA nanoswitch-nucleic acid complex having a first conformation, wherein the first conformation is characterized as locked;
contacting the DNA nanoswitch-nucleic acid complex with a biological specimen comprising one or more ribonucleases to change the first conformation to a second conformation within a mixture,
processing a mixture under conditions sufficient to separate the first conformation and the second conformation; and
contacting the first conformation and second conformation with an indicator under conditions sufficient to form a signal.

17. The method of claim 16, wherein the signal is predetermined to show a presence or absence of ribonuclease.

18. The method of claim 16, wherein the signal is predetermined to indicate a code.

19. The method of claim 16, further comprising replacing the ribonucleic acid with a deoxyribonucleic acid.

20. A polynucleotide, comprising:
a DNA nanoswitch-nucleic acid complex comprising a deoxyribonucleic acid (DNA) nanoswitch and a first oligonucleotide, wherein the DNA nanoswitch-nucleic acid complex has a first conformation characterized as locked, and a second conformation characterized as open when in a presence of ribonuclease.


SEQUENCE LISTING (144 sequences)

SEQ ID NO: 1 - DNA, 60 bp, Artificial Sequence
  Synthetic sequence
agagcataaagctaaatcggttgtaccaaaaacattatgaccctgtaatacttttgcggg

SEQ ID NO: 2 - DNA, 60 bp, Artificial Sequence
  Synthetic sequence
agaagcctttatttcaacgcaaggataaaaatttttagaaccctcatatattttaaatgc

SEQ ID NO: 3 - DNA, 60 bp, Artificial Sequence
  Synthetic sequence
aatgcctgagtaatgtgtaggtaaagattcaaaagggtgagaaaggccggagacagtcaa

SEQ ID NO: 4 - DNA, 60 bp, Artificial Sequence
  Synthetic Sequence
atcaccatcaatatgatattcaaccgttctagctgataaattaatgccggagagggtagc

SEQ ID NO: 5 - DNA, 60 bp, Artificial Sequence
  Synthetic sequence
tatttttgagagatctacaaaggctatcaggtcattgcctgagagtctggagcaaacaag

SEQ ID NO: 6 - DNA, 60 bp, Artificial Sequence
  Synthetic Sequence
agaatcgatgaacggtaatcgtaaaactagcatgtcaatcatatgtaccccggttgataa

SEQ ID NO: 7 - DNA, 60 bp, Artificial Sequence
  Synthetic Sequence
tcagaaaagccccaaaaacaggaagattgtataagcaaatatttaaattgtaaacgttaa

SEQ ID NO: 8 - DNA, 60 bp, Artificial Sequence
  Synthetic Sequence
tattttgttaaaattcgcattaaatttttgttaaatcagctcattttttaaccaatagga

SEQ ID NO: 9 - DNA, 60 bp, Artificial Sequence
  Synthetic sequence
acgccatcaaaaataattcgcgtctggccttcctgtagccagctttcatcaacattaaat

SEQ ID NO: 10 - DNA, 60 bp, Artificial Sequence
  synthetic sequence
ggataggtcacgttggtgtagatgggcgcatcgtaaccgtgcatctgccagtttgagggg

SEQ ID NO: 11 - DNA, 60 bp, Artificial Sequence
  Synthetic Sequence
acgacgacagtatcggcctcaggaagatcgcactccagccagctttccggcaccgcttct

SEQ ID NO: 12 - DNA, 60 bp, artificial sequence
  Synthetic sequence
ggtgccggaaaccaggcaaagcgccattcgccattcaggctgcgcaactgttgggaaggg

SEQ ID NO: 13 - DNA, 60 bp, Artificial sequence
  Synthetic sequence
cgatcggtgcgggcctcttcgctattacgccagctggcgaaagggggatgtgctgcaagg

SEQ ID NO: 14 - DNA, 60 bp, Artificial Sequence
  Synthetic sequence
cgattaagttgggtaacgccagggttttcccagtcacgacgttgtaaaacgacggccagt

SEQ ID NO: 15 - DNA, 60 bp, Artificial Sequence
  Synthetic sequence
gccaagcttgcatgcctgcaggtcgactctagaggatccccgggtaccgagctcgaattc

SEQ ID NO: 16 - DNA, 60 bp, artificial sequence
  synthetic sequences
gtaatcatggtcatagctgtttcctgtgtgaaattgttatccgctcacaattccacacaa

SEQ ID NO: 17 - DNA, 60 bp, Artificial Sequence
  Synthetic sequence
catacgagccggaagcataaagtgtaaagcctggggtgcctaatgagtgagctaactcac

SEQ ID NO: 18 - DNA, 60 bp, Artificial Sequence
  synthetic sequence
attaattgcgttgcgctcactgcccgctttccagtcgggaaacctgtcgtgccagctgca

SEQ ID NO: 19 - DNA, 60 bp, Artificial Sequence
  synthetic sequence
ttaatgaatcggccaacgcgcggggagaggcggtttgcgtattgggcgccagggtggttt

SEQ ID NO: 20 - DNA, 60 bp, Artificial Sequence
  synthetic sequence
gttgcagcaagcggtccacgctggtttgccccagcaggcgaaaatcctgtttgatggtgg

SEQ ID NO: 21 - DNA, 60 bp, Artificial Sequence
  synthetic sequence
ttccgaaatcggcaaaatcccttataaatcaaaagaatagcccgagatagggttgagtgt

SEQ ID NO: 22 - DNA, 60 bp, Artificial Sequence
  synthetic sequence
tgttccagtttggaacaagagtccactattaaagaacgtggactccaacgtcaaagggcg

SEQ ID NO: 23 - DNA, 60 bp, Artificial Sequence
  synthetic sequence
aaaaaccgtctatcagggcgatggcccactacgtgaaccatcacccaaatcaagtttttt

SEQ ID NO: 24 - DNA, 60 bp, Artificial Sequence
  synthetic sequence
ggggtcgaggtgccgtaaagcactaaatcggaaccctaaagggagcccccgatttagagc

SEQ ID NO: 25 - DNA, 60 bp, Artificial Sequence
  synthetic sequence
ttgacggggaaagccggcgaacgtggcgagaaaggaagggaagaaagcgaaaggagcggg

SEQ ID NO: 26 - DNA, 60 bp, Artificial Sequence
  synthetic sequence
cgctagggcgctggcaagtgtagcggtcacgctgcgcgtaaccaccacacccgccgcgct

SEQ ID NO: 27 - DNA, 60 bp, Artificial Sequence
  synthetic sequence
taatgcgccgctacagggcgcgtactatggttgctttgacgagcacgtataacgtgcttt

SEQ ID NO: 28 - DNA, 60 bp, Artificial sequence
  synthetic sequence
cctcgttagaatcagagcgggagctaaacaggaggccgattaaagggattttagacagga

SEQ ID NO: 29 - DNA, 60 bp, Artificial sequence
  synthetic sequence
acggtacgccagaatcctgagaagtgtttttataatcagtgaggccaccgagtaaaagag

SEQ ID NO: 30 - DNA, 60 bp, Artificial Sequence
  synthetic sequence
ttgcctgagtagaagaactcaaactatcggccttgctggtaatatccagaacaatattac

SEQ ID NO: 31 - DNA, 60 bp, Artificial Sequence
  synthetic sequence
cgccagccattgcaacaggaaaaacgctcatggaaatacctacattttgacgctcaatcg

SEQ ID NO: 32 - DNA, 60 bp, Artificial Sequence
  synthetic sequence
tctgaaatggattatttacattggcagattcaccagtcacacgaccagtaataaaaggga

SEQ ID NO: 33 - DNA, 60 bp, Artificial sequence
  synthetic sequence
cattctggccaacagagatagaacccttctgacctgaaagcgtaagaatacgtggcacag

SEQ ID NO: 34 - DNA, 60 bp, Artificial Sequence
  synthetic sequence
acaatatttttgaatggctattagtctttaatgcgcgaactgatagccctaaaacatcgc

SEQ ID NO: 35 - DNA, 60 bp, Artificial Sequence
  synthetic sequence
cattaaaaataccgaacgaaccaccagcagaagataaaacagaggtgaggcggtcagtat

SEQ ID NO: 36 - DNA, 60 bp, Artificial Sequence
  synthetic sequence
taacaccgcctgcaacagtgccacgctgagagccagcagcaaatgaaaaatctaaagcat

SEQ ID NO: 37 - DNA, 60 bp, Artificial Sequence
  synthetic sequence
caccttgctgaacctcaaatatcaaaccctcaatcaatatctggtcagttggcaaatcaa

SEQ ID NO: 38 - DNA, 60 bp, Artificial Sequence
  synthetic sequence
cagttgaaaggaattgaggaaggttatctaaaatatctttaggagcactaacaactaata

SEQ ID NO: 39 - DNA, 60 bp, Artificial Sequence
  synthetic sequence
gattagagccgtcaatagataatacatttgaggatttagaagtattagactttacaaaca

SEQ ID NO: 40 - DNA, 60 bp, Artificial Sequence
  synthetic sequence
cattatcattttgcggaacaaagaaaccaccagaaggagcggaattatcatcatattcct

SEQ ID NO: 41 - DNA, 60 bp, Artificial Sequence
  synthetic sequence
gattatcagatgatggcaattcatcaatataatcctgattgtttggattatacttctgaa

SEQ ID NO: 42 - DNA, 60 bp, Artificial Sequence
  synthetic sequence
taatggaagggttagaacctaccatatcaaaattatttgcacgtaaaacagaaataaaga

SEQ ID NO: 43 - DNA, 60 bp, Artificial Sequence; identical to SEQ ID NO: 42
  synthetic sequence

SEQ ID NO: 44 - DNA, 60 bp, Artificial Sequence
  synthetic sequence
cgggagaaacaataacggattcgcctgattgctttgaataccaagttacaaaatcgcgca

SEQ ID NO: 45 - DNA, 60 bp, Artificial Sequence
  synthetic sequence
gaggcgaattattcatttcaattacctgagcaaaagaagatgatgaaacaaacatcaaga

SEQ ID NO: 46 - DNA, 60 bp, Artificial Sequence
  synthetic sequence
aaacaaaattaattacatttaacaatttcatttgaattaccttttttaatggaaacagta

SEQ ID NO: 47 - DNA, 60 bp, Artificial Sequence
  synthetic sequence
cataaatcaatatatgtgagtgaataaccttgcttctgtaaatcgtcgctattaattaat

SEQ ID NO: 48 - DNA, 60 bp, Artificial Sequence
  synthetic sequence
tttcccttagaatccttgaaaacatagcgatagcttagattaagacgctgagaagagtca

SEQ ID NO: 49 - DNA, 60 bp, Artificial Sequences
  synthetic sequences
atagtgaatttatcaaaatcataggtctgagagactacctttttaacctccggcttaggt

SEQ ID NO: 50 - DNA, 60 bp, Artificial Sequence
  synthetic sequence
gaaaactttttcaaatatattttagttaatttcatcttctgacctaaatttaatggtttg

SEQ ID NO: 51 - DNA, 60 bp, Artificial Sequence
  synthetic sequence
aaataccgaccgtgtgataaataaggcgttaaataagaataaacaccggaatcataatta

SEQ ID NO: 52 - DNA, 60 bp, Artificial Sequence
  synthetic sequence
ctagaaaaagcctgtttagtatcatatgcgttatacaaattcttaccagtataaagccaa

SEQ ID NO: 53 - DNA, 60 bp, Artificial Sequence
  synthetic sequence
cgctcaacagtagggcttaattgagaatcgccatatttaacaacgccaacatgtaattta

SEQ ID NO: 54 - DNA, 60 bp, Artificial Sequence
  synthetic sequence
ggcagaggcattttcgagccagtaataagagaatataaagtaccgacaaaaggtaaagta

SEQ ID NO: 55 - DNA, 60 bp, Artificial Sequence
  synthetic sequence
attctgtccagacgacgacaataaacaacatgttcagctaatgcagaacgcgcctgttta

SEQ ID NO: 56 - DNA, 60 bp, Artificial Sequence
  synthetic sequence
tcaacaatagataagtcctgaacaagaaaaataatatcccatcctaatttacgagcatgt

SEQ ID NO: 57 - DNA, 60 bp, Artificial Sequence
  synthetic sequence
agaaaccaatcaataatcggctgtctttccttatcattccaagaacgggtattaaaccaa

SEQ ID NO: 58 - DNA, 60 bp, Artificial Sequence
  synthetic sequence
gtaccgcactcatcgagaacaagcaagccgtttttattttcatcgtaggaatcattaccg

SEQ ID NO: 59 - DNA, 60 bp, Artificial Sequence
  synthetic sequence
cgcccaatagcaagcaaatcagatatagaaggcttatccggtattctaagaacgcgaggc

SEQ ID NO: 60 - DNA, 60 bp, Artificial Sequence
  synthetic sequence
attttgcacccagctacaattttatcctgaatcttaccaacgctaacgagcgtctttcca

SEQ ID NO: 61 - DNA, 60 bp, Artificial Sequence
  synthetic sequence
gagcctaatttgccagttacaaaataaacagccatattatttatcccaatccaaataaga

SEQ ID NO: 62 - DNA, 60 bp, Artificial Sequence
  synthetic sequence
aacgattttttgtttaacgtcaaaaatgaaaatagcagcctttacagagagaataacata

SEQ ID NO: 63 - DNA, 60 bp, Artificial Sequence
  synthetic sequence
aaaacagggaagcgcattagacgggagaattaactgaacaccctgaacaaagtcagaggg

SEQ ID NO: 64 - DNA, 60 bp, Artificial Sequence
  synthetic sequence
taattgagcgctaatatcagagagataacccacaagaattgagttaagcccaataataag

SEQ ID NO: 65 - DNA, 60 bp, Artificial Sequence
  synthetic sequence
agcaagaaacaatgaaatagcaatagctatcttaccgaagccctttttaagaaaagtaag

SEQ ID NO: 66 - DNA, 60 bp, Artificial Sequence
  synthetic sequence
cagatagccgaacaaagttaccagaaggaaaccgaggaaacgcaataataacggaatacc

SEQ ID NO: 67 - DNA, 60 bp, Artificial Sequence
  synthetic sequence
caaaagaactggcatgattaagactccttattacgcagtatgttagcaaacgtagaaaat

SEQ ID NO: 68 - DNA, 60 bp, Artificial Sequence
  synthetic sequence
acatacataaaggtggcaacatataaaagaaacgcaaagacaccacggaataagtttatt

SEQ ID NO: 69 - DNA, 60 bp, Artificial sequence
  synthetic sequence
ttgtcacaatcaatagaaaattcatatggtttaccagcgccaaagacaaaagggcgacat

SEQ ID NO: 70 - DNA, 60 bp, Artificial Sequence
  synthetic sequence
tcaccgtcaccgacttgagccatttgggaattagagccagcaaaatcaccagtagcacca

SEQ ID NO: 71 - DNA, 60 bp, Artificial Sequence
  synthetic sequence
ttaccattagcaaggccggaaacgtcaccaatgaaaccatcgatagcagcaccgtaatca

SEQ ID NO: 72 - DNA, 60 bp, Artificial sequence
  synthetic sequence
gtagcgacagaatcaagtttgcctttagcgtcagactgtagcgcgttttcatcggcattt

SEQ ID NO: 73 - DNA, 60 bp, Artificial Sequence
  synthetic sequence
tcggtcatagcccccttattagcgtttgccatcttttcataatcaaaatcaccggaacca

SEQ ID NO: 74 - DNA, 60 bp, Artificial Sequence
  synthetic sequence
gagccaccaccggaaccgcctccctcagagccgccaccctcagaaccgccaccctcagag

SEQ ID NO: 75 - DNA, 60 bp, Artificial Sequence
  synthetic sequence
ccaccaccctcagagccgccaccagaaccaccaccagagccgccgccagcattgacagga

SEQ ID NO: 76 - DNA, 60 bp, Artificial Sequence
  synthetic sequence
ggttgaggcaggtcagacgattggccttgatattcacaaacaaataaatcctcattaaag

SEQ ID NO: 77 - DNA, 60 bp, Artificial Sequence
  synthetic sequence
ccagaatggaaagcgcagtctctgaatttaccgttccagtaagcgtcatacatggctttt

SEQ ID NO: 78 - DNA, 60 bp, Artificial Sequence
  synthetic sequence
gatgatacaggagtgtactggtaataagttttaacggggtcagtgccttgagtaacagtg

SEQ ID NO: 79 - DNA, 60 bp, Artificial Sequence
  synthetic sequence
cccgtataaacagttaatgccccctgcctatttcggaacctattattctgaaacatgaaa

SEQ ID NO: 80 - DNA, 60 bp, Artificial Sequence
  synthetic sequence
ccaggcggataagtgccgtcgagagggttgatataagtatagcccggaataggtgtatca

SEQ ID NO: 81 - DNA, 60 bp, Artificial Sequence
  synthetic sequence
ccgtactcaggaggtttagtaccgccaccctcagaaccgccaccctcagaaccgccaccc

SEQ ID NO: 82 - DNA, 60 bp, Artificial Sequence
  synthetic sequence
tcagagccaccaccctcattttcagggatagcaagcccaataggaacccatgtaccgtaa

SEQ ID NO: 83 - DNA, 60 bp, Artificial Sequence
  synthetic sequence
cactgagtttcgtcaccagtacaaactacaacgcctgtagcattccacagacagccctca

SEQ ID NO: 84 - DNA, 60 bp, Artificial Sequence
  synthetic sequence
tagttagcgtaacgatctaaagttttgtcgtctttccagacgttagtaaatgaattttct

SEQ ID NO: 85 - DNA, 60 bp, Artificial Sequence
  synthetic sequence
gtatgggattttgctaaacaactttcaacagtttcagcggagtgagaatagaaaggaaca

SEQ ID NO: 86 - DNA, 60 bp, Artificial Sequence
  synthetic sequence
actaaaggaattgcgaataataattttttcacgttgaaaatctccaaaaaaaaggctcca

SEQ ID NO: 87 - DNA, 60 bp, Artificial Sequence
  Synthetic sequence
aaaggagcctttaattgtatcggtttatcagcttgctttcgaggtgaatttcttaaacag

SEQ ID NO: 88 - DNA, 60 bp, Artificial sequence
  synthetic sequence
cttgataccgatagttgcgccgacaatgacaacaaccatcgcccacgcataaccgatata

SEQ ID NO: 89 - DNA, 60 bp, Artificial Sequence
  Synthetic sequence
ttcggtcgctgaggcttgcagggagttaaaggccgcttttgcgggatcgtcaccctcagc

SEQ ID NO: 90 - DNA, 60 bp, Artificial Sequence
  synthetic sequence
ctttttcatgaggaagtttccattaaacgggtaaaatacgtaatgccactacgaaggcac

SEQ ID NO: 91 - DNA, 60 bp, Artificial Sequence
  synthetic sequence
caacctaaaacgaaagaggcaaaagaatacactaaaacactcatctttgacccccagcga

SEQ ID NO: 92 - DNA, 60 bp, Artificial Sequence
  synthetic sequence
ttataccaagcgcgaaacaaagtacaacggagatttgtatcatcgcctgataaattgtgt

SEQ ID NO: 93 - DNA, 60 bp, Artificial Sequence
  synthetic sequence
cgaaatccgcgacctgctccatgttacttagccggaacgaggcgcagacggtcaatcata

SEQ ID NO: 94 - DNA, 60 bp, Artificial Sequence
  synthetic sequence
agggaaccgaactgaccaactttgaaagaggacagatgaacggtgtacagaccaggcgca

SEQ ID NO: 95 - DNA, 60 bp, Artificial sequence
  synthetic sequence
taggctggctgaccttcatcaagagtaatcttgacaagaaccggatattcattacccaaa

SEQ ID NO: 96 - DNA, 60 bp, Artificial Sequence
  synthetic sequence
tcaacgtaacaaagctgctcattcagtgaataaggcttgccctgacgagaaacaccagaa

SEQ ID NO: 97 - DNA, 60 bp, Artificial Sequence
  synthetic sequence
cgagtagtaaattgggcttgagatggtttaatttcaactttaatcattgtgaattacctt

SEQ ID NO: 98 - DNA, 60 bp, Artificial Sequence
  synthetic sequence
atgcgattttaagaactggctcattataccagtcaggacgttgggaagaaaaatctacgt

SEQ ID NO: 99 - DNA, 60 bp, Artificial Sequence
  Synthetic sequence
taataaaacgaactaacggaacaacattattacaggtagaaagattcatcagttgagatt

SEQ ID NO: 100 - DNA, 60 bp, Artificial sequence
  synthetic sequence
taagagcaacactatcataaccctcgtttaccagacgacgataaaaaccaaaatagcgag

SEQ ID NO: 101 - DNA, 60 bp, Artificial sequence
  synthetic sequence
aggcttttgcaaaagaagttttgccagagggggtaatagtaaaatgtttagactggatag

SEQ ID NO: 102 - DNA, 60 bp, Artificial sequence
  synthetic sequence
cgtccaatactgcggaatcgtcataaatattcattgaatccccctcaaatgctttaaaca

SEQ ID NO: 103 - DNA, 60 bp, Artificial Sequence
  synthetic sequence
gttcagaaaacgagaatgaccataaatcaaaaatcaggtctttaccctgactattatagt

SEQ ID NO: 104 - DNA, 60 bp, Artificial Sequence
  synthetic sequence
cagaagcaaagcggattgcatcaaaaagattaagaggaagcccgaaagacttcaaatatc

SEQ ID NO: 105 - DNA, 60 bp, Artificial sequence
  synthetic sequence
gcgttttaattcgagcttcaaagcgaaccagaccggaagcaaactccaacaggtcaggat

SEQ ID NO: 106 - DNA, 60 bp, Artificial sequence
  synthetic sequence
tagagagtacctttaattgctccttttgataagaggtcatttttgcggatggcttagagc

SEQ ID NO: 107 - DNA, 60 bp, Artificial sequence
  synthetic sequence
ttaattgctgaatataatgctgtagctcaacatgttttaaatatgcaactaaagtacggt

SEQ ID NO: 108 - DNA, 60 bp, Artificial sequence
  synthetic sequence
gtctggaagtttcattccatataacagttgattcccaattctgcgaacgagtagatttag

SEQ ID NO: 109 - DNA, 49 bp, Artificial sequence
  synthetic sequence
tttgaccattagatacatttcgcaaatggtcaataacctgtttagctat

SEQ ID NO: 110 - DNA, 60 bp, Artificial Sequence
  Synthetic sequence
aacatccaataaatcatacaggcaaggcaaagaattagcaaaattaagcaataaagcctc

SEQ ID NO: 111 - DNA, 60 bp, Artificial Sequence
  synthetic sequence
gtgagcgagtaacaacccgtcggattctccgtgggaacaaacggcggattgaccgtaatg

SEQ ID NO: 112 - DNA, 60 bp, Artificial Sequence
  Synthetic sequence
ttcttttcaccagtgagacgggcaacagctgattgcccttcaccgcctggccctgagaga

SEQ ID NO: 113 - DNA, 60 bp, Artificial Sequence
  Synthetic sequence
agcgaaagacagcatcggaacgagggtagcaacggctacagaggctttgaggactaaaga

SEQ ID NO: 114 - DNA, 60 bp, Artificial Sequence
  synthetic sequence
taggaataccacattcaactaatgcagatacataacgccaaaaggaattacgaggcatag

SEQ ID NO: 115 - DNA, 60 bp, Artificial Sequence
  Synthetic sequence
attttcatttggggcgcgagctgaaaaggtggcatcaattctactaatagtagtagcatt

SEQ ID NO: 116 - DNA, 60 bp, Artificial Sequence
  synthetic sequence
tctgtccatcacgcaaattaaccgttgtagcaatacttctttgattagtaataacatcac

SEQ ID NO: 117 - DNA, 60 bp, Artificial Sequence
  synthetic sequence
attcgacaactcgtattaaatcctttgcccgaacgttattaattttaaaagtttgagtaa

SEQ ID NO: 118 - DNA, 60 bp, Artificial Sequence
  synthetic sequence
tgggttatataactatatgtaaatgctgatgcaaatccaatcgcaagacaaagaacgcga

SEQ ID NO: 119 - DNA, 60 bp, Artificial Sequence
  Synthetic sequence
gttttagcgaacctcccgacttgcgggaggttttgaagccttaaatcaagattagttgct

SEQ ID NO: 120 - DNA, 60 bp, Artificial Sequence
  Synthetic sequence
tcaaccgattgagggagggaaggtaaatattgacggaaattattcattaaaggtgaatta

SEQ ID NO: 121 - DNA, 60 bp, Artificial Sequence
  Synthetic sequence
gtattaagaggctgagactcctcaagagaaggattaggattagcggggttttgctcagta

SEQ ID NO: 122 - DNA, 20 bp, Artificial sequence
  synthetic sequence
tctgtccatcacgcaaatta

SEQ ID NO: 123 - DNA, 20 bp, Artificial Sequence
  Synthetic sequence
aattttaaaagtttgagtaa

SEQ ID NO: 124 - DNA, 20 bp, Artificial Sequence
  Synthetic sequence
tcgcaagacaaagaacgcga

SEQ ID NO: 125 - DNA, 20 bp, Artificial Sequence
  synthetic sequence
ttaaatcaagattagttgct

SEQ ID NO: 126 - DNA, 20 bp, Artificial Sequence
  Synthetic sequence
tattcattaaaggtgaatta

SEQ ID NO: 127 - DNA, 20 bp, Artificial Sequence
  Synthetic sequence
tagcggggttttgctcagta

SEQ ID NO: 128 - DNA, 51 bp, Artificial Sequence
  synthetic sequence
accgttgtagcaatacttctttgattagtaataacatcaccaagctgattt

SEQ ID NO: 129 - DNA, 51 bp, Artificial Sequence
  Synthetic sequence
acacccggtgaattcgacaactcgtattaaatcctttgcccgaacgttatt

SEQ ID NO: 130 - DNA, 51 bp, Artificial Sequence
  Synthetic sequence
accgttgtagcaatacttctttgattagtaataacatcaccgccaatattt

SEQ ID NO: 131 - DNA, 51 bp, Artificial Sequence
  synthetic sequence
acgtgctgctatgggttatataactatatgtaaatgctgatgcaaatccaa

SEQ ID NO: 132 - DNA, 50 bp, Artificial Sequence
  Synthetic sequence
accgttgtagcaatacttctttgattagtaataacatcacccaacaacat

SEQ ID NO: 133 - DNA, 51 bp, Artificial Sequence
  Synthetic sequence
gaaactacctagttttagcgaacctcccgacttgcgggaggttttgaagcc

SEQ ID NO: 134 - DNA, 51 bp, Artificial Sequence
  Synthetic sequence
accgttgtagcaatacttctttgattagtaataacatcacaaccacacaac

SEQ ID NO: 135 - DNA, 51 bp, Artificial Sequence
  synthetic sequence
ctactacctcatcaaccgattgagggagggaaggtaaatattgacggaaat

SEQ ID NO: 136 - DNA, 51 bp, Artificial Sequence
  Synthetic sequence
accgttgtagcaatacttctttgattagtaataacatcacccacacacttc

SEQ ID NO: 137 - DNA, 51 bp, Artificial Sequence
  Synthetic sequence
cttacattccagtattaagaggctgagactcctcaagagaaggattaggat

SEQ ID NO: 138 - RNA, 22 nt, Artificial Sequence
  Synthetic sequence
ucaccggguguaaaucagcuug

SEQ ID NO: 139 - RNA, 22 nt, Artificial Sequence
  synthetic sequence
uagcagcacguaaauauuggcg

SEQ ID NO: 140 - RNA, 21 nt, Artificial Sequence
  synthetic sequence
uagguaguuucauguuguugg

SEQ ID NO: 141 - RNA, 22 nt, Artificial Sequence
  Synthetic sequence
ugagguaguagguugugugguu

SEQ ID NO: 142 - RNA, 22 nt, Artificial Sequence
  synthetic sequence
uggaauguaaggaagugugugg

SEQ ID NO: 143 - DNA, 22 bp, Artificial Sequence
  Synthetic sequence
tgaggtagtaggttgtgtggtt

SEQ ID NO: 144 - DNA, 40 bp, Artificial Sequence
  synthetic sequence
ctactaatagtagtagcattaacatccaataaatcataca